(19) United States
(12) Patent Application Publication
(10) Pub. No.: US 2010/0278865 A1
(43) Pub. Date: Nov. 4, 2010

(54) PRODUCTION AND USE OF EPITOPE-TAGGED HEPATITIS C VIRUS PARTICLE

(76) Inventors: Takaji Wakita, Tokyo (JP); Tetsuro Suzuki, Tokyo (JP); Hitoshi Takahashi, Tokyo (JP)

Correspondence Address: BIRCH STEWART KOLASCH & BIRCH, PO BOX 747, FALLS CHURCH, VA, 22040-0747, US

(21) Appl. No.: 12/668,861

(22) PCT Filed: Jul. 11, 2008
(86) PCT No.: PCT/JP2008/062977

§ 371 Date: Jan. 12, 2010

(30) Foreign Application Priority Data
Jul. 13, 2007 (JP) 2007-184587

(51) Int. Cl. A61K 39/29; C07H 21/04; C12N 15/79; C12N 7/00; C12N 5/071; C12N 7/02; C07K 16/08; A61P 31/14

(52) U.S. Cl. 424/228.1; 536/23.72; 435/320.1; 435/235.1; 435/370; 435/239; 530/389.4

(57) ABSTRACT

This invention relates to a nucleic acid comprising a nucleic acid sequence encoding an epitope tag peptide in the hypervariable region 1 of the E2 protein of naturally occurring or chimeric hepatitis C virus (HCV) genome, an epitope-tagged HCV particle encoded by such nucleic acid, and a method for purifying the HCV particles at a high purity in a simple manner with the use of an anti-epitope tag antibody-immobilized support.

### TECHNICAL FIELD

The present invention relates to epitope-tagged hepatitis C virus particles, a vector used for producing such viruses, and a cell line that produces such virus particles. The present invention also relates to a method for purifying such virus particles, a vaccine obtained by inactivating such virus particles, and an anti-hepatitis C virus antibody to such virus particles.

### BACKGROUND ART

Hepatitis C virus (which may be simply referred to as “HCV” hereinafter) was found and identified as a major causative virus of non-A and non-B hepatitis (Choo, Q L. et al., Science, 244: 359-362, 1989), and highly sensitive methods for detecting HCV had been established. Thus, the number of new HCV patients caused by blood transfusion has dramatically decreased. However, the number of virus carriers in Japan is deduced to be over 2,000,000 and over 170,000,000 in the world, including so-called virus carriers who have not yet developed hepatitis symptoms. This is mainly because the rate of chronicity of hepatitis due to HCV infection is as high as 70-80% and because there are no effective antiviral agents other than interferon at present. Further, hepatitis C is a serious infection with poor prognosis, and approximately half of the patients with chronic hepatitis C would unexceptionally experience worsening of symptoms from hepatitis C to cirrhosis and hepatic cancer. Accordingly, development of antiviral agents or vaccines aimed at preventing virus carriers from developing the disease or at eliminating viruses has been awaited.

In order to develop therapeutic agents for HCV, a cell culture system used for studying infectious HCV particles or a mechanism of HCV particle growth is needed. In the past, an available experimentation system that can effectively grow HCV was limited to an animal experimentation system using chimpanzees, which constitutes a major cause for delayed development of therapeutic agents for HCV. In 2005, however, Wakita et al. developed a method for preparing infectious viruses in a cell culture system (WO 05080575 A1, WO 06022422 A1, and Wakita, T. et al., Nat. Med., 11:791-796, 2005), which enabled development of therapeutic agents for HCV using infectious HCV particles.

Under the above circumstances, there are needs for development of a novel technique that can purify infectious HCV particles in a more simple manner with higher purity than conventional techniques for purifying infectious HCV particles, which were produced in a culture cell system (WO 06022422 A1 and Zahn, A. & Allain1, J. P., J. Gen. Virol., 86: 677-685, 2005).

As the method for purifying a protein obtained in a cell culture system at a high purity in a simple manner, a method wherein a protein of interest is expressed as a fusion protein with protein label and the fusion protein is purified with the use of an antibody to the protein label or a molecule that specifically binds thereto, is known.

The protein label is referred to as an epitope tag or simply as a tag. Examples of known epitope tags include FLAG peptide, 3×FLAG peptide, HA peptide, 3×HA peptide, myc peptide, 6×His peptide, GST polypeptide, MBP polypeptide, PDZ domain polypeptide, tandem affinity purification (TAP) peptide, alkaline phosphatase, and avidin. Such peptides or polypeptides are generally fused to the N- or C-terminus of the protein of interest, and such peptides or polypeptides can be optionally inserted into the protein of interest.

When an epitope tag is added to a protein, however, a biological activity or conformation inherent to the protein might not be always maintained, depending on the type of a protein, the type of an epitope tag, a site of a protein to which an epitope tag is added, or the like. Accordingly, it is necessary to study biological functions of epitope-tagged proteins, for every type of proteins, for every type of epitope tags, or for every site to which an epitope tag is added.

The HCV genome is a single-stranded (+) RNA comprising approximately 9,600 nucleotides. This genomic RNA comprises 5′-untranslated region (also referred to as “5′ NTR” or “5′ UTR”), a translational region composed of structural regions and non-structural regions, and 3′-untranslated region (also referred to as “3′ NTR” or “3′ UTR”). In the structural regions, HCV structural proteins are encoded and, in the non-structural regions, a plurality of non-structural proteins are encoded.

Such HCV structural proteins (Core, E1, E2, and p7) and non-structural proteins (NS2, NS3, NS4A, NS4B, NS5A, and NS5B) are first translated as a series of polyproteins from the translational region, subjected to limited degradation by protease, and then released. Among structural proteins, Core is a core protein, and E1 and E2 are envelope proteins. It is known that non-structural proteins are associated with replication of the virus itself, NS2 has metalloprotease activity, and NS3 has serine protease activity (one third of the N-terminal side) and helicase activity (two thirds of the C-terminal side). Further, it is reported that NS4A is a cofactor for protease activity of NS3, and NS5B has RNA-dependent RNA polymerase activity.

HCV is coated with a coat called envelope. The envelope comprises a component derived from the membrane of a host cell and proteins derived from the virus. The proteins that constitute the HCV envelope comprise envelope protein 1 (referred to as “E1”), envelope protein 2 (referred to as “E2”), and p7. In particular, E1 and E2 each have a transmembrane region at the C terminus, E1 and E2 are anchored to the HCV membrane through such transmembrane region, and E1 and E2 are associated with HCV particle formation or infection (Voisset, C. & Dubuisson, Biology of the Cell, 96: 413-420, 2004; and Op De Beeck, A. et al., J. Gen. Virol., 82: 2589-2595, 2001).

There are two regions, at the amino acid terminal side of the E2 protein, having significantly different sequences among virus strains, and they are named hypervariable region 1 (referred to as HVR1) and hypervariable region 2 (referred to as HVR2) (Hijikata, M. et al., Biochem Biophys Res Commun., 175: 220-228, 1991). HVR1 comprises 27 amino acid residues at the amino terminus of the E2 protein, and it is exposed on the surface of the E2 protein, serving as a B cell epitope. While amino acid sequence diversity of HVR1 is 80% at maximum, there are several regions in which amino acids are preserved. Thus, it is considered that a given secondary structure is necessary for virus survival. Further, there are substantially no cysteine residues or no signals for addition of asparagine-binding sugar chains in HVR1. Meanwhile, HVR2 comprises 7 amino acids and it is located in the region at positions 91-97 from the amino terminus of the E2 protein. While HVR2 exists in the genome of the HCV 1b type, it does not exist in HCV of other genotypes.

As an example of HCV in which epitope tags have been inserted into virus particles, the JFH-1 strain (type HCV2a) in which HVR1 of the E2 protein has been substituted with HA peptide is known, and it is confirmed to have infectivity (Wakita, T. et al., Nat. Med., 11: 791-796, 2005). However, there is no report showing that such infectious epitope-tagged HCV particles are highly purified.

### DISCLOSURE OF THE INVENTION

The present invention is intended to provide epitope-tagged HCV particles that can be simply purified at a high purity, a method for purifying the same, and a vaccine prepared using such highly purified HCV particles.

The present inventors have studied epitope tag types, which would not affect productivity and infectivity when used for cell culture of HCV particles, and combinations of a HCV protein to which an epitope tag is added with a site of addition, thereby succeeding in attaining the first object of the present invention; i.e., preparation of epitope-tagged HCV particles. Further, the present inventors prepared clones, which produce HCV particles at high levels, and confirmed that the prepared epitope-tagged HCV particles could be highly purified via a single step with the use of an anti-epitope tag antibody column. This has led to the completion of the present invention.

Specifically, the present invention includes (1) to (15) below.

(1) A nucleic acid comprising (a), (b), or (c) below and comprising a nucleic acid sequence encoding an epitope tag peptide at hypervariable region 1 (HVR1) in an E2 protein coding sequence of the (a), (b), or (c):

(a) the genome of hepatitis C virus (HCV) JFH-1 strain;

(b) a chimeric hepatitis C virus (HCV) genome comprising: 5′-untranslated region, a Core protein coding sequence, an E1 protein coding sequence, an E2 protein coding sequence, and a p7 protein coding sequence of HCV J6CF strain; and a NS2 protein coding sequence, a NS3 protein coding sequence, a NS4A protein coding sequence, a NS4B protein coding sequence, a NS5A protein coding sequence, a NS5B protein coding sequence, and 3′-untranslated region of the HCV JFH-1 strain; or

(c) a chimeric hepatitis C virus (HCV) genome comprising: 5′-untranslated region of the JFH-1 strain; a Core protein coding sequence, an E1 protein coding sequence, an E2 protein coding sequence, and a p7 protein coding sequence of HCV TH1 strain; and a NS2 protein coding sequence, a NS3 protein coding sequence, a NS4A protein coding sequence, a NS4B protein coding sequence, a NS5A protein coding sequence, a NS5B protein coding sequence, and 3′-untranslated region of the HCV JFH-1 strain.

(2) The nucleic acid according to (1), wherein the epitope tag peptide comprises the amino acid sequence DYKDDDDK or DYKDHDGDYKDHDIDYKDDDDK.

(3) The nucleic acid according to (1) or (2), wherein the epitope tag peptide further comprises a linker sequence.

(4) The nucleic acid according to (3), wherein the linker sequence comprises GGG sequence, GGGGS sequence, or (GGGGS)₃ sequence, or a sequence comprising deletion, substitution or addition of 1-10 amino acids in a part of the sequence GGG, GGGGS or (GGGGS)₃.

(5) The nucleic acid according to any one of (1) to (4), which is shown in SEQ ID NO: 11, 12, 30, 31, 41, or 42, wherein nucleotide “T” shown in the sequence listing is read as “U” when the nucleic acid is RNA.

(6) The nucleic acid according to any one of (1) to (5), which, when the amino acid subsequent to the end of the sequence of the hypervariable region 1 (HVR1) is designated as an amino acid at position 1, comprises a mutation which is a substitution of asparagine at position 122 or 124 with lysine.

(7) The nucleic acid according to (6), which is shown in SEQ ID NO: 20 or 21 wherein nucleotide “T” shown in the sequence listing is read as “U” when the nucleic acid is RNA.

(8) A vector comprising the nucleic acid according to any one of (1) to (7).

(9) An epitope-tagged hepatitis C virus (HCV) particle comprising the nucleic acid according to any one of (1) to (7) as the genome.

(10) A cell comprising the epitope-tagged hepatitis C virus (HCV) particle according to (9).

(11) The cell according to (10), which is Huh7 or a cell line derived therefrom.

(12) A method for purifying epitope-tagged hepatitis C virus (HCV) particle comprising:

a step of preparing epitope-tagged HCV particles from the cell according to (10) or (11);

a step of bringing a solution containing the epitope-tagged HCV particles into contact with an anti-epitope tag antibody-immobilized support and then adsorbing the epitope-tagged HCV particles to the anti-epitope tag antibody-immobilized support; and

a step of releasing the epitope-tagged HCV particles from the anti-epitope tag antibody-immobilized support to obtain the epitope-tagged HCV particles.

(13) The method of purification according to (12), wherein the releasing step comprises releasing the epitope-tagged HCV particles from the anti-epitope tag antibody-immobilized support using any of a buffer containing epitope tag peptide, a buffer containing no calcium, 0.1M glycine buffer (pH 2.5-4.0), 1M arginine HCl buffer (pH 4.0-5.0), or 0.1-1 mM HCl.

(14) An hepatitis C virus (HCV) vaccine, which is obtained by inactivating the epitope-tagged HCV particle according to (9).

(15) An anti-hepatitis C virus (HCV) antibody to the epitope-tagged HCV particle according to (9).

This description includes all or part of the contents as disclosed in the description and/or drawings of Japanese Patent Application No. 2007-184587, which is a priority document of the present application.

### BRIEF DESCRIPTION OF THE DRAWINGS

FIG. 1 shows the structure of HCV E2. N1 to N11 each represent a sugar chain addition site, HVR represents a hypervariable region, and TMD represents a transmembrane domain. Part of the amino acid sequence of E2 HVR1 of J6/JFH-1 (the underlined portion) was removed, and the FLAG-tag sequence (the underlined portion) was inserted therein.

FIG. 2A, FIG. 2B, and FIG. 2C each show the structures of pJ6/JFH-1 (1×FLAG) and pJ6/JFH-1 (3×FLAG) plasmids. FIG. 2B is a continuation of FIG. 2A and FIG. 2C is a continuation of FIG. 2B.

FIG. 3 shows changes in the amount of the HCV Core proteins in the culture supernatant after J6/JFH-1 RNA and J6/JFH-1 (3×FLAG) RNA were introduced into the Huh-7 cells. The amount of the produced Core protein did not change when J6/JFH-1 was introduced. When J6/JFH-1 (3×FLAG) was introduced, the amount of the produced Core protein decreased until day 22 after introduction, but thereafter increasing to an almost same level as that of J6/JFH-1 on day 35.

FIG. 4 shows the expression of HCV Core and E2 proteins on days 4, 17, 30 and 43 after J6/JFH-1 RNA or J6/JFH-1 (3×FLAG) RNA were introduced into Huh-7 cells. When J6/JFH-1 (3×FLAG) was introduced, the molecular masses of the E2 protein on days 30 and 43 were found to be lower than that on day 4.

FIG. 5 shows behavior of the HCV E2 protein which was treated with endoglycosidase (PNGase F) on days 4, 17, 30 and 43 after J6/JFH-1 (3×FLAG) RNA was introduced the Huh-7 cells. By treating the protein with endoglycosidase, differences in molecular mass as observed in FIG. 4 were no longer observed.

FIG. 6 shows the results of the sequence analysis of the HCV genome 8 and 39 days after introduction of J6/JFH-1 (3×FLAG) RNA. Asparagine (N6), which is a sugar chain addition site in the E2 region, was already substituted with lysine (K) 39 days after RNA introduction.

FIG. 7 shows the amount of HCV Core protein in the solution of purified J6/JFH-1 (3×FLAG) virus. Since HCV Core protein was detected after elution of 1×FLAG peptide or 3×FLAG peptide, purification of HCV particles was confirmed.

FIG. 8 shows contamination of proteins contained in the solution of purified J6/JFH-1 (3×FLAG) virus. The solution of the purified virus contained almost no contaminant proteins.

FIG. 9 shows infectivity of purified J6/JFH-1 (3×FLAG) virus. HCV RNA in the cells was quantified 4 days after infection with viruses. The purified virus was found to have infectivity equivalent to that of unpurified virus.

FIG. 10A, FIG. 10B, and FIG. 10C each show the structures of pJ6/JFH-1 (1×FLAG) N→K and pJ6/JFH-1 (3×FLAG) N→K plasmids. FIG. 10B is a continuation of FIG. 10A and FIG. 10C is a continuation of FIG. 10B.

FIG. 11 shows the structure of HCV E2. N1 to N11 each represent a sugar chain addition site, HVR represents a hypervariable region, and TMD represents a transmembrane domain. Part of the amino acid sequence of E2 HVR1 of JFH-1 (the underlined portion) was removed, and the FLAG-tag sequence (the underlined portion) was inserted therein.

FIG. 12A, FIG. 12B, and FIG. 12C each show the structures of pJFH-1 (1×FLAG) and pJFH-1 (3×FLAG) plasmids. FIG. 12B is a continuation of FIG. 12A and FIG. 12C is a continuation of FIG. 12B.

FIG. 13 shows the structure of HCV E2. N1 to N11 each represent a sugar chain addition site, HVR represents a hypervariable region, and TMD represents a transmembrane domain. Part of the amino acid sequence of E2 HVR1 of pTH/JFH-1 (the underlined portion) was removed, and the FLAG-tag sequence (the underlined portion) was inserted therein.

FIG. 14A, FIG. 14B, and FIG. 14C each show the structures of pTH/JFH-1 (1×FLAG) and pTH/JFH-1 (3×FLAG) plasmids. FIG. 14B is a continuation of FIG. 14A and FIG. 14C is a continuation of FIG. 14B.

### BEST MODES FOR CARRYING OUT THE INVENTION

When implementing the present invention, conventional molecular biological and immunological techniques within the technical scope in the art are to be utilized. Such techniques are obvious to those skilled in the art and have already been thoroughly reported in the published documents (see, for example, Sambrook et al., Molecular Cloning: A Laboratory Manual, vol. 3, 2001; Ed Harlow et al., Antibodies: A Laboratory Manual, 1988).

All publications, patents, and patent applications cited herein are incorporated herein by reference in their entirety.

### 1. Epitope-Tagged HCV Particles

The term “epitope-tagged HCV particle” as used herein refers to a HCV particle in which an epitope tag peptide has been added in a HCV structural protein. Such epitope-tagged HCV particle can be produced in a cell culture system, and such particle preferably has infectivity.

Production of HCV particles in a cell culture system requires the use of a full-length gene derived from the JFH-1 strain or a chimera gene of the JFH-1 strain and another HCV strain. The fundamental technique thereof is described in WO 04104198 A1, WO 06022422 A1, Wakita, T. et al., Nat. Med. 11: 791-796, 2005, and Lindenbach, B D. et al., Science 309: 623-626, 2005.

Specifically, an embodiment of the gene that can produce HCV particles is the JFH-1 virus genomic RNA comprising 5′-untranslated region, a Core protein coding sequence, an E1 protein coding sequence, an E2 protein coding sequence, a p7 protein coding sequence, a NS2 protein coding sequence, a NS3 protein coding sequence, a NS4A protein coding sequence, a NS4B protein coding sequence, a NS5A protein coding sequence, a NS5B protein coding sequence, and 3′-untranslated region, in order in the 5′→3′ direction.

Another embodiment of the gene that can generate HCV particles is a chimeric gene comprising virus genomic RNAs of 2 or more types of HCV strains. An example of such RNA is RNA comprising 5′-untranslated region, a Core protein coding sequence, an E1 protein coding sequence, an E2 protein coding sequence, a p7 protein coding sequence, and a NS2 protein coding sequence of an HCV strain other than the JFH-1; and a NS3 protein coding sequence, a NS4A protein coding sequence, a NS4B protein coding sequence, a NS5A protein coding sequence, a NS5B protein coding sequence, and 3′-untranslated region of the JFH-1 strain, in order in the 5′→′ direction.

Specific examples of genes that can generate HCV particles include the nucleic acids (a), (b), and (c) below:

(a) the genome of the JFH1 strain (i.e., a nucleic acid comprising 5′-untranslated region, a Core protein coding sequence, an E1 protein coding sequence, an E2 protein coding sequence, a p7 protein coding sequence, a NS2 protein coding sequence, a NS3 protein coding sequence, a NS4A protein coding sequence, a NS4B protein coding sequence, a NS5A protein coding sequence, a NS5B protein coding sequence, and 3′-untranslated region of the JFH-1 strain, in order in the 5′→3′ direction (see SEQ ID NO: 30: pJFH-1 (1×FLAG), SEQ ID NO: 31: pJFH-1 (3×FLAG));

(b) a chimeric HCV genome comprising: 5′-untranslated region, a Core protein coding sequence, an E1 protein coding sequence, an E2 protein coding sequence, and a p7 protein coding sequence of J6CF strain; and a NS2 protein coding sequence, a NS3 protein coding sequence, a NS4A protein coding sequence, a NS4B protein coding sequence, a NS5A protein coding sequence, a NS5B protein coding sequence, and 3′-untranslated region of the JFH-1 strain, in order in the 5′→3′ direction (preferably a chimeric HCV genome comprising: 5′-untranslated region, a Core protein coding sequence, an E1 protein coding sequence, an E2 protein coding sequence, a p7 protein coding sequence, and a sequence encoding the N-terminal 16 amino acids of the NS2 protein encoding region; and a sequence encoding a region from the N-terminal 17^{th} amino acid to the C terminus of the NS2 protein encoding region, a NS3 protein coding sequence, a NS4A protein coding sequence, a NS4B protein coding sequence, a NS5A protein coding sequence, a NS5B protein coding sequence, and 3′-untranslated region of the JFH-1 strain, in order in the 5′→3′ direction (see SEQ ID NO: 10: pJ6/JFH-1, SEQ ID NO: 11: pJ6/JFH-1 (1×FLAG), SEQ ID NO: 12: pJ6/JFH-1 (3×FLAG)); and

(c) a chimeric HCV genome comprising: 5′-untranslated region of the JFH1 strain; a Core protein coding sequence, an E1 protein coding sequence, an E2 protein coding sequence, and a p7 protein coding sequence of TH1 strain; and a NS2 protein coding sequence, a NS3 protein coding sequence, a NS4A protein coding sequence, a NS4B protein coding sequence, a NS5A protein coding sequence, a NS5B protein coding sequence, and 3′-untranslated region of the JFH-1 strain, in order in the 5′→3′ direction (preferably, a chimeric HCV genome comprising: 5′-untranslated region of the JFH-1 strain; a Core protein coding sequence, an E1 protein coding sequence, an E2 protein coding sequence, a p7 protein coding sequence, and a sequence encoding the N-terminal 33 amino acids of the NS2 protein encoding region of the TH strain; and a sequence encoding a region from the N-terminal 34^{th} amino acid to the C terminus of the NS2 protein encoding region, a NS3 protein coding sequence, a NS4A protein coding sequence, a NS4B protein coding sequence, a NS5A protein coding sequence, a NS5B protein coding sequence, and 3′-untranslated region of the JFH-1 strain, in order in the 5′→3′ direction (see SEQ ID NO: 40: pTH/JFH1, SEQ ID NO: 41: pTH/JFH1 (1×FLAG), SEQ ID NO: 42: pTH/JFH1 (3×FLAG)).

In the present invention, a nucleic acid sequence encoding an epitope tag peptide, i.e. DYKDDDDKGGG (SEQ ID NO: 49) or DYKDHDGDYKDHDIDYKDDDDKGGG (SEQ ID NO: 50), is inserted in frame into the hypervariable region 1 (HVR1) of the E2 protein coding sequence of the above HCV genome. Alternatively, part of said region is substituted with said epitope tag peptide to prepare epitope-tagged HCV genome DNA. The “epitope-tagged HCV particles” are produced in a cell culture system using such DNA to obtain highly purified epitope-tagged HCV particles.

### 2. Preparation of Epitope-Tagged HCV Particle

(1) Preparation of HCV Genome DNA having an Epitope Tag in the HCV Structural Protein

The term “epitope tag” (or simply referred to as “tag”) as used herein is not particularly limited, provided that it can be used for labeling HCV. Examples thereof include the FLAG peptide (which may be referred to as the flag peptide or Flag peptide), the 3×FLAG peptide (which may be referred to as the 3×FLAG peptide, 3×Flag peptide, or 3×flag peptide), the HA peptide, the 3×HA peptide, the myc peptide, the 6×His peptide, the GST polypeptide, the MBP polypeptide, the PDZ domain polypeptide, the tandem affinity purification (TAP) peptide, alkaline phosphatase, and avidin. In the present invention, the FLAG peptide (the amino acid sequence: DYKDDDDK (SEQ ID NO: 51)) or the 3×FLAFG peptide (the amino acid sequence: DYKDHDGDYKDHDIDYKDDDDK (SEQ ID NO: 52)) is used as the epitope tag peptide, and among them, the 3×FLAG peptide is particularly preferable.

The position at which the epitope tag is added is a HVR1 region of the E2 protein of HCV particle. The epitope tag peptide is inserted in frame (i.e., so that the reading frame is not misaligned) into a region between the amino acids at positions 8 and 19 counted from the N-terminus of the E2 protein having low homology between HCV strains, in the HVR1 region, or alternatively, preferably the epitope tag peptide is inserted into the position at which the N-terminal amino acids 11-17 have been removed.

Where necessary, a suitable linker may be added to the C-terminus of the epitope tag peptide. Used as the linker are the amino acid sequences GGG (SEQ ID NO: 53), GGGGS (SEQ ID NO: 54), and (GGGGS)×3 (also referred to as (GGGGS)₃), or peptides comprising a deletion, substitution, or addition of about 1-10, preferably 1-5, more preferably 1-2 amino acids in part of said amino acid sequences. Preferable examples are GGG, GGGGS (SEQ ID NO: 54), and (GGGGS)×3, preferably GGG.

An example of a method for introducing a nucleic acid encoding the epitope tag into the HCV genome is a method wherein polymerase chain reaction (PCR) is carried out using synthetic primers containing a nucleic acid sequence encoding the epitope tag and cDNA of the HCV genome as a template, and the resulting PCR product is adequately subjected to restriction enzyme treatment and ligation treatment to recombine the above cDNA into cDNA of the HCV genome of interest. Examples of such procedures are described in Examples below.

HCV strains used are, but not limited to, strains belonging to any of 6 genotypes classified by lineage analysis using the nucleotide sequences of the HCV strains: genotype 1a (e.g., H77 strain (GenBank Accession No. AF011751)), genotype 1b (e.g., J1 strain (GenBank Accession No. D89815), Con1 strain (GenBank Accession No. AJ238799, which may be referred to as the strain Con-1 or con1), TH strain (Wakita, T. et al., J. Biol. Chem., 269, 14205-14210, 1994, JP Patent Publication (kokai) No. 2004-179 A)), genotype 2a (e.g., the JFH-1 strain (GenBank Accession No. AB047639, which may be referred to as “JFH1 strain”), the J6CF strain (GenBank Accession No. AF177036), JCH-1 (GenBank Accession No. AB047640), JCH-2 (GenBank Accession No. AB047641), JCH-3 (GenBank Accession No. AB047642), JCH-4 (GenBank Accession No. AB047643), JCH-5 (GenBank Accession No. AB047644), JCH-6 (GenBank Accession No. AB047645)), genotype 2b (e.g., HC-J8 strain (GenBank Accession No. D01221)), genotype 3a (e.g., NZL1 strain (GenBank Accession No. D17763)), and genotype 3b (e.g., Tr-Kj (GenBank Accession No. D49374)). In the Examples later, a nucleic acid sequence comprising the non-structural protein-encoding sequences and 3′-untranslated region derived from JFH-1 and the 5′-untranslated region and structural protein-encoding sequences derived from TH1, JFH-1, or J6CF, is used as a preferable example of the present invention.

To insert an epitope tag peptide of interest into HVR of HCV, a cDNA of the HCV genomic RNA is cloned into a vector, and PCR is carried out using the vector as a template and using synthetic DNAs encoding an epitope tag peptide as primers. Thus, the cDNA encoding an envelope protein in which an epitope tag has been added to or inserted into a target site, can be obtained.

Furthermore, a cDNA fragment encoding an envelope protein, which has an epitope tag, is isolated after digestion with a suitable restriction enzyme, cDNA of full-length HCV genomic RNA is cloned into a site downstream of a promoter such as a T7 promoter to prepare a vector, which is then digested with the same restriction enzyme as above, and a cDNA fragment encoding an envelope protein, which has the isolated epitope tag sequence, is inserted at the same digestion site. Thereby being able to obtain a full-length HCV cDNA.

The vector into which the cDNA of full-length HCV genomic RNA as used herein has been cloned is capable of producing HCV particles, when the RNA transcribed from the vector is introduced into HCV permissive cells, such as Huh7 cell. The details regarding the structure and preparation thereof are described in WO 04104198 A1, WO 06022422 A1, Wakita, T. et al., Nat. Med. 11: 791-796, 2005, and Lindenbach, B D. et al., Science 309: 623-626, 2005.

### (2) Production of Epitope-Tagged HCV Particles in Cell Culture System

The RNA may be synthesized from a HCV cDNA having an epitope tag coding sequence under the control of a promoter, which RNA is introduced into a cell to obtain a cell containing epitope-tagged HCV particles, and subsequently the obtained cells are cultured to prepare epitope-tagged HCV particles. The promoter includes, but is not limited to, T7 promoter, SP6 promoter, and T3 promoter, preferably T7 promoter.

To prepare the RNA in vitro using, as a template, a nucleic acid into which a HCV cDNA has been introduced under control of T7 promoter and then cloned, a kit such as MEGAscript T7 kit (Ambion) can be employed.

Cells for introducing the RNA are any cells capable of producing HCV particles, such as Huh7, HepG2, IMY-N9, HeLa or 293 cells, or cells which express CD81 and/or Claudin1 genes and are derived from Huh7, HepG2, IMY-N9, HeLa or 293 cells. Among such cells, Huh7 cell or Huh7-derived cell lines are preferably used. Examples of the Huh7-derived cell lines include Huh7.5 and Huh7.5.1.

Examples of methods for introducing the RNA into cells include calcium phosphate coprecipitation, a DEAE-dextran method, lipofection, microinjection, and electroporation. Lipofection and electroporation are preferable, and electroporation is more preferable.

A cDNA may be introduced into cells instead of RNA. In such a case, the HCV cDNA to which an epitope tag coding sequence has been added is operably inserted into a vector comprising an RNA polymerase I promoter and a terminator (Neumann, G. et al., Virology, 202: 477-479, 1994, WO 2007/037428 A1), and the vector is introduced into cells in the same manner as in the case of RNA to express the cDNA in the cells.

The capacity of the cells for virus particle production can be evaluated by use of antibodies to proteins that constitute a HCV particle released in culture solution, such as a Core protein, an E1 protein, and an E2 protein. Also, the capacity of the cells for HCV virus particle production may be indirectly evaluated by amplifying a HCV genomic RNA contained in the HCV virus particles in a culture medium via RT-PCR using specific primers, to detect the RNA.

Whether or not the prepared viruses are infectious can be evaluated by culturing cells into which HCV RNA has been introduced, treating HCV permissive cells (e.g., Huh7) with the supernatant from said culture, and immunologically staining the cells with an anti-Core antibody 48 hours later to count the number of infected cells. Alternatively, the evaluation can be carried out by subjecting the cell extract to electrophoresis on SDS-polyacrylamide gel and detecting Core proteins via Western blotting.

### (3) Obtaining Epitope-Tagged HCV Particles-Producing Cell Lines

For efficient replication of the HCV genome, it is necessary that a mutation occurs in the nucleotide sequence of the genome (Lohmann, V. et al., J. Virol. 75: 1437-1449, 2001). Mutation that enhances replication is referred to as “adaptive mutation.” The cells into which the HCV genomic RNA has been introduced as prepared in (2) above may be subjected to subculture to obtain cell lines that continuously produce HCV particles. When such culture is continued, there is a case where adaptive mutation takes place in the HCV genome, resulting in significantly enhancing the production of HCV particles.

When an amino acid subsequent to the end of the HVR1 sequence (Puntoriero, G. et al., EMBO J. 17: 3521-3533, 1998) in the nucleic acids (a) to (c) is designated as the 1^{st} amino acid, the mutation which is a substitution of asparagine, a 122^{nd} or 124^{th} amino acid, with lysine is suitable as a mutation when producing epitope-tagged HCV particles. In the case of the nucleic acid (a) or (b), particularly, the mutation which is a substitution of asparagine, a 124^{th} amino acid, with lysine is more preferable. In the nucleic acid (c), the mutation which is a substitution of asparagine, a 122^{nd} amino acid, with lysine is more preferable. Asparagine at these positions is considered to be modified with a sugar chain, and it is suggested that there is a correlation between no addition of a sugar chain and high productivity of HCV particles.

In the present invention, the adaptive mutation can be attained via subculture as described above, or can be artificially introduced. When it is artificially introduced, mutagenic primers may be designed, and PCR may be carried out using such primers to synthesize DNA into which mutation has been introduced. Also, the commercially available Quick Change II XL Site-Directed Mutagenesis kit (Stratagene) may be used to prepare a mutant.

In the present invention, it is difficult to detect a nucleic acid mutation that is necessary for mutating one amino acid, by hybridization techniques; however, the adaptive mutation can be confirmed by determining the sequence of a HCV gene.

(4) Purification of Epitope-Tagged HCV Particles

Epitope-tagged HCV particles are purified using a support to which a protein capable of binding to the epitope tag has been bound. The most general protein is an antibody to epitope tag peptide. In the present invention, a preferable epitope tag is FLAG or 3×FLAG peptide, and a support to which an anti-FLAG antibody capable of binding to said peptide, such as an M2 antibody or M5 antibody, has been bound (or immobilized) (available from Sigma) or the like can be used.

A culture supernatant of FLAG epitope-tagged HCV particle-producing cells or 3×FLAG epitope-tagged HCV particle-producing cells or a solution containing HCV particles is applied to a column containing the anti-FLAG antibody-bound support, then the column is washed with phosphate buffer, and HCV particles bound to the anti-FLAG antibody-bound support can be eluted with a buffer containing the FLAG or 3×FLAG peptide. When a support to which an M5 antibody that binds to the FLAG peptide in a calcium-dependent manner has been bound is used, HCV particles are bound with the use of a calcium-containing buffer, and HCV particles can be eluted from the support with the use of a calcium free buffer.

As other elution techniques, general techniques for eluting proteins from antibody-bound supports can be employed (Antibodies: A Laboratory Manual, Cold Spring Harbor Laboratory, 1988). For example, 0.1M glycine buffer (pH 2.5-4.0) or 1M arginine HCl buffer (pH 4.0-5.0) can be used as an elution solution (Ejima, D. et al., Anal. Biochem. 345: 250-257, 2005). Further, 0.1 mM-1 mM hydrochloric acid can be used. To prevent the eluted HCV particles from denaturation, the pH of the buffer after elution is preferably set at around neutral (pH 6.0-8.0).

The thus-purified HCV particles are subjected to electrophoresis on SDS-polyacrylamide gel and HCV-derived proteins are detected by Western blot, whereby it can be determined whether or not the HCV particles are purified.

### 3. Utilization of Epitope-Tagged HCV Particles

### (1) Vaccine

Highly purified epitope-tagged HCV particles are preferably used as a vaccine or as an antigen for preparing anti-HCV antibodies.

Specifically, the prepared epitope-tagged HCV particles can be inactivated by a method known in the art for use thereof. The virus can be inactivated by adding and mixing an inactivator such as formalin, β-propiolactone, or glutardialdehyde in, for example, a virus suspension and allowing the inactivator to react with the virus (Appaiahgari et al., Vaccine, 22: 3669-3675, 2004).

Furthermore, the virus may be irradiated with ultraviolet to eliminate infectivity of virus, thereby being immediately inactivated. Irradiation of ultraviolet leads to inactivation of the virus with little influence on constituted proteins. A source of ultraviolet rays used for inactivation can be a commercially available germicidal lamp, particularly a 15w germicidal lamp, although the source is not limited thereto. In the present invention, HCV particles that are purified by the method described above are used, and methods of inactivation are not limited by a purified or unpurified state. Preferably, a solution containing epitope-tagged HCV particles may be irradiated with ultraviolet at 20 mW/cm² at room temperature for at least 5 minutes to inactivate epitope-tagged HCV particles.

The vaccine of the present invention is in the form of a solution or suspension, containing 10%-95%, preferably 25%-70% active ingredient (virus particles or part thereof), and is formulated for administration. The vaccine can be prepared in a solid state that is suitable for dissolving or suspending it in a solution. The preparation may be emulsified or may be encapsulated in liposomes.

An active immunogenic ingredient, such as HCV particle, is often mixed with a pharmaceutically acceptable excipient which is compatible with the active ingredient. Examples of adequate excipients include water, physiological saline, dextrose, glycerol, ethanol, and mixtures thereof. Further, the vaccine can contain a minor amount of an auxiliary agent(s) (e.g., a humidifier or emulsifier), a pH buffer, and/or an adjuvant that enhances vaccine efficacy, where needed. Examples of the effective adjuvant include, but are not limited to, aluminum hydroxide, N-acetyl-muramyl-L-threonyl-D-isoglutamine (thr-MDP), N-acetyl-nor-muramyl-L-alanyl-D-isoglutamine (referred to as CGP11637 or nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (referred to as CGP19835A or MTP-PE), and RIBI. RIBI contains three components extracted from bacteria; i.e. monophosphoryl lipid A, trehalose dimycolate, and a cell wall skeleton (HPL+TDM+CWS), in 2% squalene/Tween® 80 emulsion. Efficacy of an adjuvant can be determined by assaying the amount of antibodies resulting from administration of a vaccine comprising HCV particles.

The vaccine of the present invention may generally be administered parenterally, by injection such as subcutaneous injection or intramuscular injection, for example. Examples of other formulations that are suitable as other dosage forms for administration include suppositories and, optionally, oral preparations.

If necessary, one or more compounds having adjuvant activity can be added to the HCV vaccine. An adjuvant is a non-specific stimulant to the immune system. Such substance enhances the immune response of a host against HCV vaccines. Specific examples of adjuvants that are known in the art include Freund's complete and incomplete adjuvants, vitamin E, a nonionic block copolymer, muramyl dipeptide, saponin, mineral oil, vegetable oil, and Carbopol. Examples of adjuvants that are particularly suitable for mucosal application include E. coli thermolabile toxin (LT) and Cholera toxin (CT). Examples of other adequate adjuvants include aluminum hydroxide, aluminum phosphate or aluminum oxide, an oil emulsion (e.g., Bayol® or Marcol 52®), saponin, and a vitamin E solubilizate. Accordingly, the vaccine of a preferable embodiment of the present invention comprises an adjuvant.

For example, injections for subcutaneous, intracutaneous, intramuscular, or intravenous administration can be administered, which injections comprise the vaccine of the invention and a pharmaceutically acceptable carrier or diluent, in combination with others such as a stabilizer, a carbohydrate (e.g., sorbitol, mannitol, starch, sucrose, glucose, or dextran), a protein (e.g., albumin or casein), a protein-containing substance (e.g., bovine serum or skimmed milk), and buffer (e.g., phosphate buffer).

The vaccine of the present invention is administered in a manner suitable for a dosage form and in an amount that can exert preventive and/or therapeutic effects. The amount to be administered is generally 0.01 μg-100,000 μg antigen per dose. Such amount varies depending on patients to be treated, the capacity of patients for antibody synthesis in the immune system, and the desired degree of protection. Also, the amount varies depending on the route of administration, such as oral, subcutaeous, intracutaneous, intramuscular, or intravenous administration.

The vaccine of the present invention can be administered according to a single-administration schedule, and preferably according to a multiple-administration schedule. In the multiple-administration schedule, 1-10 separate administrations are performed at the time of initiation of inoculation, and another administration can then be performed with the time interval that is necessary for maintaining and/or enhancing the immune response. For example, the second administration can be performed 1-4 months later. Where needed, administration may be subsequently performed several months later. The administration regimens are, at least partially, determined according to the necessity of an individual, and the regimens depend on the judgment made by a doctor.

The vaccine of the present invention may be administered with another immunosuppressant agent (e.g., immunoglobulin).

Further, the vaccine of the present invention may be administered to a healthy individual to induce an immune response to HCV in such healthy individual, and the vaccine may be used for preventing new HCV infection. Furthermore, the vaccine may be administered to a patient infected with HCV to induce a potent immune response to HCV in vivo, and thus the vaccine may be used as a therapeutic vaccine which eliminates HCV.

(2) Anti-HCV Antibody Induced with the Use of Epitope-Tagged HCV Particle Antigens

The epitope-tagged HCV particles of the present invention can be easily purified and are of high purity. Thus, such particles are useful as antigens for preparing antibodies.

Specifically, animal blood serum contains antibodies to impurities. When the HCV particle sample used for animal immunization contains impurities, accordingly, the serum needs to be purified using an HCV antigen column in order to eliminate antibodies to such impurities. In the present invention, however, because the epitope-tagged HCV particles are obtained as highly purified HCV, any purification step is not needed.

The anti-HCV antibody can be prepared by administering the epitope-tagged HCV particles of the present invention to mammalians or birds. Examples of mammalians include mice, rats, rabbits, goats, sheep, horses, cattle, guinea pigs, dromedaries, Bactrian camel, and lama. Dromedaries, Bactrian camel, and lama are suitable for preparing an antibody consisting of the H chain alone. Examples of birds include chickens, geese, and ostriches. The blood serum may be taken from an animal to which the particles of the present invention have been administered, in order to obtain antibodies of interest by well-known methods.

With the use of animal cells immunized with the epitope-tagged HCV particles of the present invention, hybridomas that produce monoclonal antibody-producing cells can be prepared. Methods for producing hybridomas are well-known in the art, and the method described in Antibodies: A Laboratory Manual (Cold Spring Harbor Laboratory, 1988) can be employed.

Monoclonal antibody-producing cells may be prepared via cell fusion or via other means such as introduction of an oncogene (DNA) or immortalization of B lymphocytes via infection with Epstein-Barr virus.

Monoclonal or polyclonal antibodies thus obtained are useful for diagnosis, treatment, and prevention of HCV.

The antibodies prepared using the epitope-tagged HCV particles of the present invention are used for treating or preventing hepatitis C. Further, they are used for preventing HCV infection from donor organs at the time of organ transplantation. Further, the above antibodies can be used in combination with existing antiviral agents, such as interferon and ribavirin.

An effective dose of the anti-HCV antibody can be selected from between 0.001 mg and 1,000 mg per kg body weight. Alternatively, the amount of administration may be 0.01 to 100,000 mg/patient's body. It should be noted, however, that the amount of a therapeutic agent comprising the anti-HCV antibody prepared using the epitope-tagged HCV particles of the invention is not limited to the above-mentioned amounts.

The administration route of the above therapeutic agent includes, but is not limited to, preferably subcutaneous, intracutaneous, or intramuscular administration, more preferably intravenous administration. Also, the timing for administration of the therapeutic agent is not limited and may be either before or after patients develop clinical symptoms of the disease.

The therapeutic agent comprising, as an active ingredient, the anti-HCV antibody prepared using the epitope-tagged HCV particles of the present invention can be formulated by conventional techniques (Remington's Pharmaceutical Science, Latest Edition, Mark Publishing Company, Easton, U.S.A.), in combination with pharmaceutically acceptable carriers or additives.

Examples of such carriers and pharmaceutical additives include water, pharmaceutically acceptable organic solvents, collagen, polyvinyl alcohol, polyvinyl pyrrolidone, carboxyvinyl polymer, sodium carboxymethylcellulose, sodium polyacrylate, sodium alginate, water-soluble dextran, sodium carboxymethyl starch, pectin, methylcellulose, ethyl cellulose, xanthan gum, gum Arabic, casein, agar, polyethylene glycol, diglycerine, glycerine, propylene glycol, vaseline, paraffin, stearyl alcohol, stearic acid, human serum albumin (HSA), mannitol, sorbitol, lactose, and surfactants acceptable as pharmaceutical additives.

Actually employed additives are selected from among the above-mentioned additives depending upon dosage forms of the therapeutic agent of the invention, and such additives can be used alone or optionally in combination, although the additives, of course, are not limited to the above-mentioned additives. When the additive is used for an injection preparation, for example, the anti-HCV antibody prepared using the purified epitope-tagged HCV particles is dissolved in a solvent, such as physiological saline, buffer, or a glucose solution, followed by addition of an adsorption preventing agent, such as Tween 80, Tween 20, gelatin, or human serum albumin. Alternatively, the additive may be lyophilized to be prepared in a dosage form dissolved and reconstituted before use. Examples of excipients for lyophilization include sugar alcohols, such as mannitol or dextrose, and sugars.

Hereafter, the present invention will be described in more detail with reference to the following examples. It should be noted that these examples are provided for illustrative purposes and the technical scope of the present invention is not limited to these examples.

### EXAMPLES

### Example 1

### Construction of J6/JFH-1 Plasmid with FLAG-Tag Sequence Inserted in HCV E2 HVR1 Region

Used as a cDNA of HCV genomic RNA was the cDNA of a J6/JFH-1 chimera comprising a genotype 2a strain J6CF-derived sequence from 5′-UTR to N-terminal 16^{th} amino acid residue of NS2 (GenBank Accession No. AF177036, Yanagi, M. et al., Virology, 262, 1999, pp. 250-263) and a genotype 2a strain JFH-1-derived sequence from N-terminal 17^{th} amino acid residue of NS2 to 3′-UTR (GenBank Accession No. AB047639, Kato, T. et al., Gastroenterology, 125, 2003, pp. 1808-1817). As the FLAG-tag, cDNA comprising 1 or 3 repeats of the FLAG sequence was inserted. The resulting plasmids are referred to as pJ6/JFH-1 (1×FLAG) and pJ6/JFH-1 (3×FLAG), respectively.

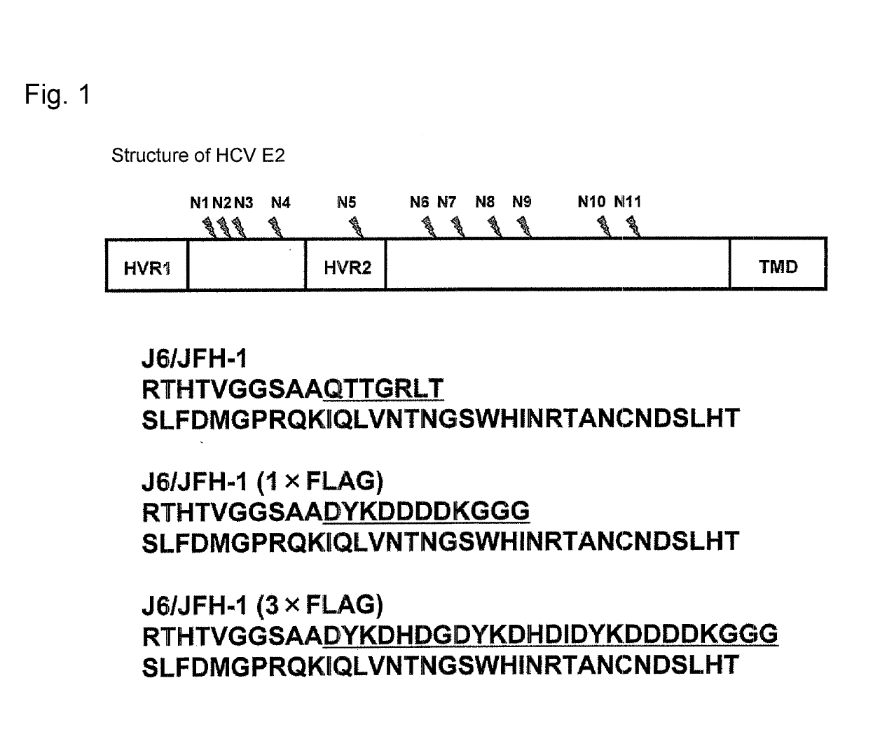

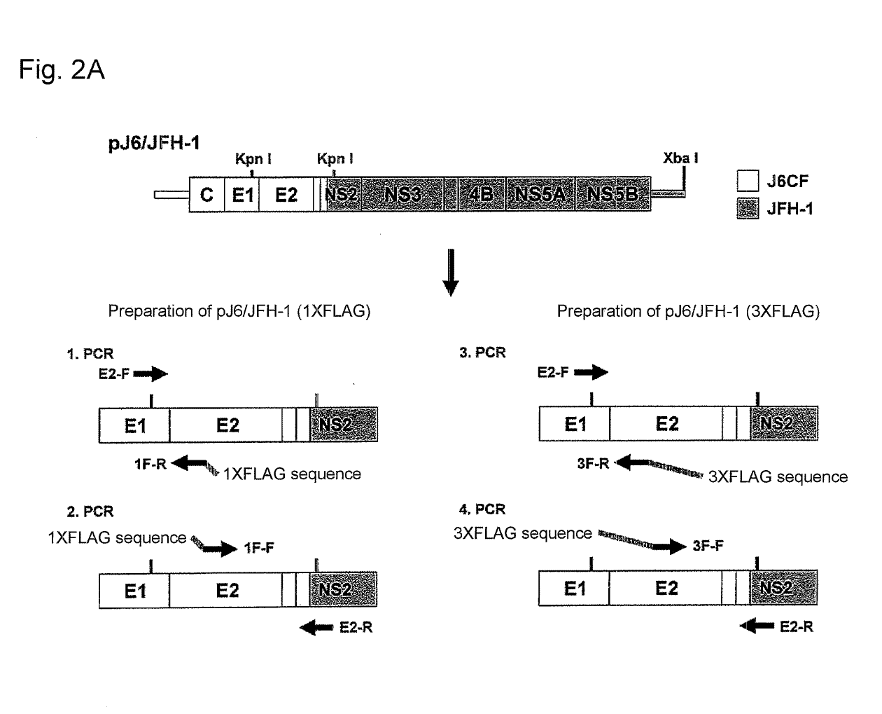

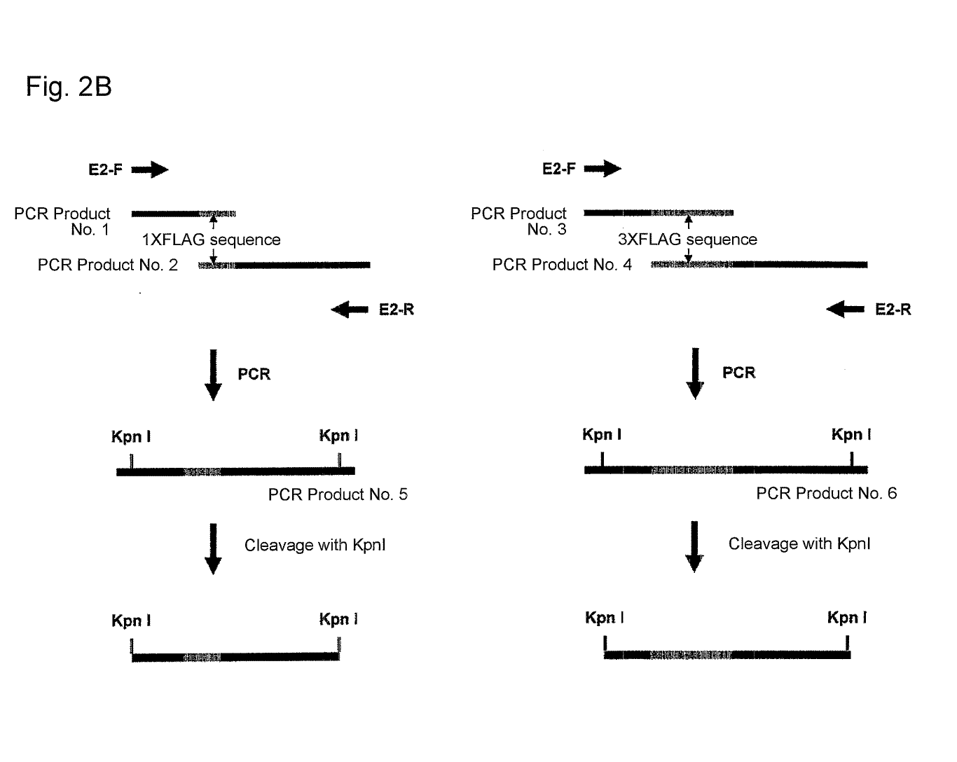

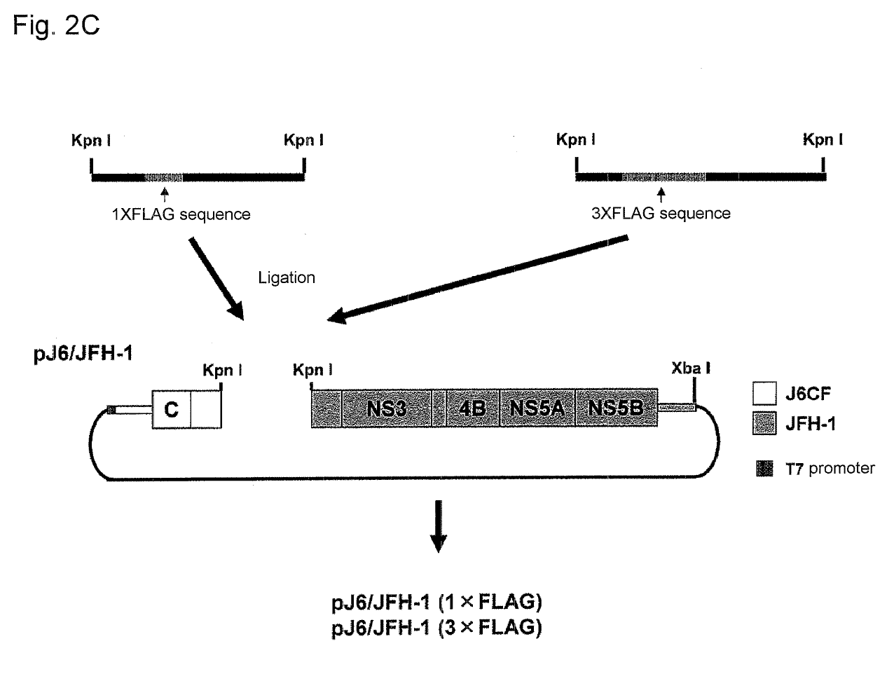

FIG. 1 shows the amino acid sequence which is in the vicinity of the FLAG tag-insertion site translated from those genes, starting from the N-terminal amino acid of the E2 protein. The N-terminal amino acid sequence of the E2 protein encoded by J6/JFH1 is shown in SEQ ID NO: 1, the N-terminal amino acid sequence of the E2 protein encoded by pJ6/JFH-1 (1×FLAG) is shown in SEQ ID NO: 2, and the N-terminal amino acid sequence of the E2 protein encoded by pJ6/JFH-1 (3×FLAG) is shown in SEQ ID NO:3. The methods for preparing plasmids are shown in FIG. 2 (A) to (C).

Specifically, cDNA corresponding to the entire JFH1 strain-derived genomic RNA region was cloned into pUC19 plasmid to construct a plasmid DNA, pJFH1 (Wakita, T. et al. Nat. Med., 11, 2005, pp. 791-796, WO 2004/104198), which was digested with EcoRI and then partially digested with BclI, and a plasmid DNA fragment from which a fragment (about 2,840 bp) from the EcoRI site to the first BclI site had been removed was purified. Separately, a J6CF strain-derived genomic cDNA which had been cloned into pUC19 plasmid, i.e. pJ6CF (GenBank Accession No. AF177036, Yanagi, M., et al., Virology 262: 250-263, 1999), was partially digested with EcoRI and BclI to obtain an about 2,840-bp fragment, which was then ligated to the above purified fragment to obtain J6/JFH1.

Subsequently, to the J6/JFH1 cDNA as a template were added 10 μl of 10×buffer, 4 μl of 2 mM dNTPs mixture, and 1 μl each of 10 μM primers E2-F (SEQ ID NO: 4) and 1F-R (SEQ ID NO: 5), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles where one cycle consists of 98° C. for 10 seconds, 55° C. for 15 seconds, and 72° C. for 30 seconds. The resulting PCR product was designated as PCR product No. 1.

To the J6/JFH1 cDNA as a template were added 10 μl of 10×buffer, 4 μl of 2 mM dNTPs mixture, and 1 μl each of 10 μM primers E2-R (SEQ ID NO: 6) and 1F-F (SEQ ID NO: 7), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles where one cycle consists of 98° C. for 10 seconds, 55° C. for 15 seconds, and 72° C. for 1.5 minutes. The resulting PCR product was designated as PCR product No. 2.

To the J6/JFH1 cDNA a template were added 10 μl of 10×buffer, 4 μl of 2 mM dNTPs mixture, and 1 μl each of 10 μM primers E2-F (SEQ ID NO: 4) and 3F-R (SEQ ID NO: 8), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles where one cycle consists of 98° C. for 10 seconds, 55° C. for 15 seconds, and 72° C. for 30 seconds. The resulting PCR product was designated as PCR product No. 3.

To the J6/JFH1 cDNA as a template were added 10 μl of 10×buffer, 4 μl of 2 mM dNTPs mixture, and 1 μl each of 10 μM primers E2-R (SEQ ID NO: 6) and 3F-F (SEQ ID NO: 9), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles where one cycle consists of 98° C. for 10 seconds, 55° C. for 15 seconds, and 72° C. for 1.5 minutes. The resulting PCR product was designated as PCR product No. 4.

Each PCR product was purified and dissolved in 15 μl of H₂O. DNA (1 μl) of the PCR product No. 1 was mixed with DNA (1 μl) of the PCR product No. 2. To the mixture, which was used as a template, were added 10 μl of 10×buffer, 4 μl of the 2 mM dNTPs mixture, and 1 μl each of 10 μM primers E2-F (SEQ ID NO: 4) and E2-R (SEQ ID NO: 6), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles where one cycle consists of 98° C. for 10 seconds, 55° C. for 15 seconds, and 72° C. for 2 minutes. The resulting PCR product was designated as PCR product No. 5.

DNA (1 μl) of the PCR product No. 3 was mixed with DNA (1 μl) of the PCR product No. 4. To the mixture, which was used as a template, were added 10 μl of 10×buffer, 4 μl of 2 mM dNTPs mixture, and 1 μl each of 10 μM primers E2-F (SEQ ID NO: 4) and E2-R (SEQ ID NO: 6), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles where one cycle consists of 98° C. for 10 seconds, 55° C. for 15 seconds, and 72° C. for 2 minutes. The resulting PCR product was designated as PCR product No. 6. The PCR products were purified and dissolved in 30 μl of H₂O.

The J6/JFH-1 cDNA and the purified PCR product No. 5 were separately digested with the KpnI restriction enzyme, and each HCV cDNA fragment was fractionated by agarose gel electrophoresis, followed by purification. The two DNA fragments were mixed with Ligation Mix (TAKARA BIO INC, Japan) to ligate the DNA fragments. The vector was designated as pJ6/JFH-1 (1×FLAG). The pJ6/JFH-1 (1×FLAG) comprises a nucleotide sequence that encodes the chimera gene, which comprises: in order from 5′-side to 3′-side, the 5′-untranslated region, the Core protein-encoding sequence, the E1 protein-coding sequence, the E2 protein-coding sequence, the p7 protein-coding sequence, and a sequence encoding the N-terminal 16 amino acids of the NS2 protein region, of the J6CF strain; and a sequence encoding a region from the N-terminal 17^{th} amino acid to the C-terminus of the NS2 protein region, the NS3 protein-coding sequence, the NS4A protein-coding sequence, the NS4B protein-coding sequence, the NS5A protein-coding sequence, the NS5B protein region, and the 3′-untranslated region, of the JFH-1 strain, wherein it comprises, in the E2 HVR1 region, a nucleotide sequence that encodes the FLAG peptide sequence.

The J6/JFH-1 cDNA and the purified PCR product No. 6 were digested with the KpnI restriction enzyme, and each HCV cDNA fragment was fractionated by agarose gel electrophoresis, followed by purification. The two DNA fragments were mixed with Ligation Mix (TAKARA BIO INC, Japan) to ligate the two DNA fragments. The obtained vector was designated as pJ6/JFH-1 (3×FLAG). The pJ6/JFH-1 (3×FLAG) comprises a nucleotide sequence that encodes the chimera gene, which comprises: in order from 5′-side to 3′-side, the 5′-untranslated region, the Core protein-coding sequence, the E1 protein-coding sequence, the E2 protein-coding sequence, the p7 protein-coding sequence, and a sequence encoding the N-terminal 16 amino acids of the NS2 protein region, of the J6CF strain; and a sequence encoding a region from the N-terminal amino acid 17 to the C-terminus of the NS2 protein region, the NS3 protein-coding sequence, the NS4A protein-coding sequence, the NS4B protein-coding sequence, the NS5A protein-coding sequence, the NS5B protein-coding region, and the 3′-untranslated region, of the JFH-1 strain, wherein it comprises, in the E2 HVR1 region, a nucleotide sequence that comprises 3 repeats of the FLAG peptide sequence.

The nucleotide sequences of pJ6/JFH-1, pJ6/JFH-1 (1×FLAG), and pJ6/JFH-1 (3×FLAG) are shown in SEQ ID NO: 10, SEQ ID NO: 11, and SEQ ID NO: 12, respectively, in the Sequence Listing.

### Example 2

### In Vitro RNA Synthesis and Introduction of RNA into Cells

The pJ6/JFH-1, pJ6/JFH-1 (1×FLAG), and pJ6/JFH-1 (3×FLAG) were separately cleaved with XbaI and subjected to phenol/chloroform extraction then to ethanol precipitation. The cleaved plasmids each was used as a template to synthesize a HCV RNA using the MEGAscript T7 kit (Ambion).

The Huh7 cells (3×10⁶ cells) and 5 μg of the HCV RNA were suspended in 400 μl of the Cytomix solution (120 mM KCl, 0.15 mM CaCl₂, 10 mM K₂HPO₄/KH₂PO₄, 25 mM Hepes, 2 mM EGTA, 5 mM MgCl₂, 20 mM ATP, and 50 mM glutathione), transferred into a 4-mm cuvette, and subjected to electroporation using the Gene Pulser (BioRad) at 260 V and 950 μF. Thereafter, the cells into which the HCV RNA had been introduced were seeded on a 10 cm² dish and then subcultured.

### Example 3

### Production of HCV Particles Using J6/JFH-1 (3×FLAG) RNA Introduced Cells

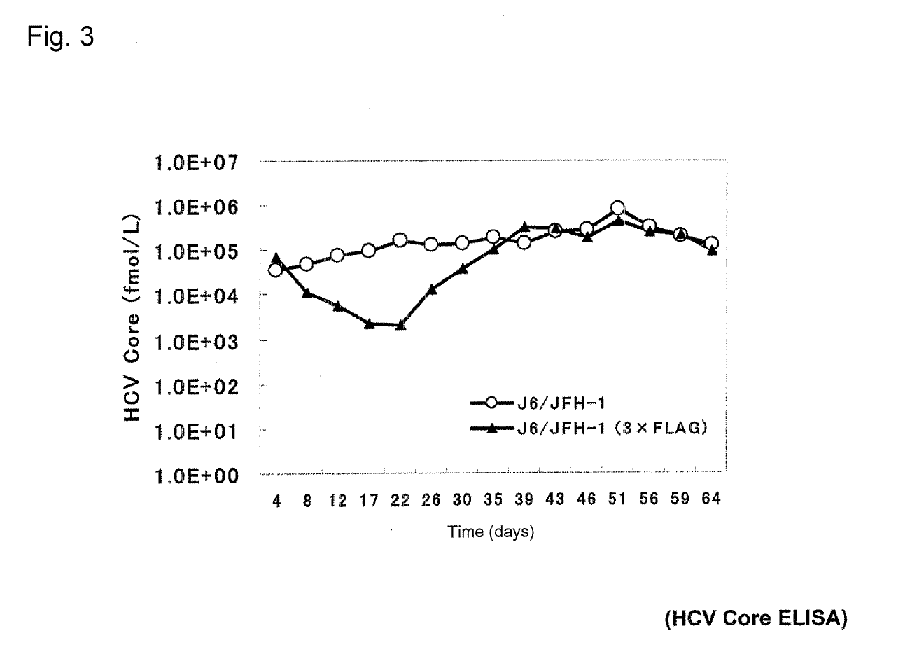

When the cells into which the J6/JFH-1 or J6/JFH-1 (3×FLAG) RNA had been introduced were subcultured, each HCV Core protein contained in the culture supernatant was quantified using the HCV antigen ELISA test kit (Ortho) to confirm the production of HCV particles. As a result, the amount of production of the HCV particles was found to remain unchanged at substantially constant levels when the J6/JFH-1 RNA was introduced. When the J6/JFH-1 (3×FLAG) RNA was introduced, however, the amount of HCV Core protein in the culture supernatant decreased with time until day 21 after the introduction, and then began to increase on day 22 after the introduction and reached an almost same level as that of J6/JFH-1 on day 35 after the introduction (FIG. 3). This suggested that, although the J6/JFH-1 (3×FLAG) RNA did not have a high capacity for virus production when the Huh7 cells were introduced, it had a capacity for virus production similar to that of J6/JFH-1 RNA after an adaptive mutation, which is necessary for virus production, was introduced into the virus genome.

### Example 4

### Expression of HCV Protein in J6/JFH-1 (3×FLAG) RNA Introduced Cells

Expression of HCV proteins in the cells was confirmed by Western blotting on days 4, 17, 30 and 43 after the introduction of J6/JFH-1 or J6/JFH-1 (3×FLAG) RNA. In this analysis, a cell extract sample obtained from the Huh7 cells with no introduced RNA was used as a negative control. Samples extracted from the cells were subjected to SDS-PAGE and blotted onto a PVDF membrane (Immobilon-P, Millipore). Thereafter, anti-Core monoclonal antibody, anti-FLAG monoclonal antibody (M2 antibody, Sigma), anti-E2 monoclonal antibody, and HRP-labeled secondary antibody that recognizes these antibodies were used to detect the Core proteins and the E2 proteins translated in the cells using the ECL Plus (GE Healthcare).

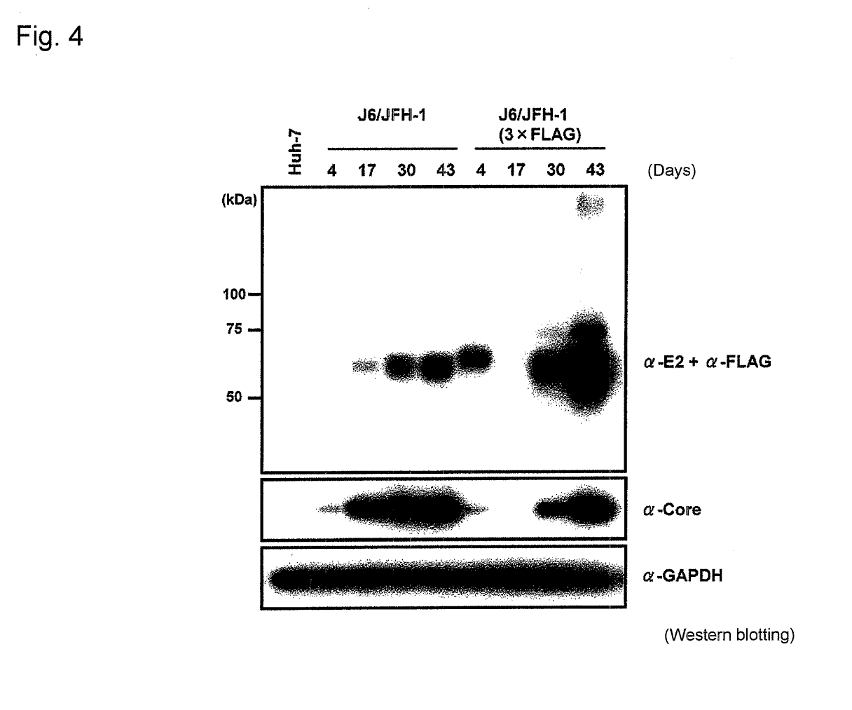

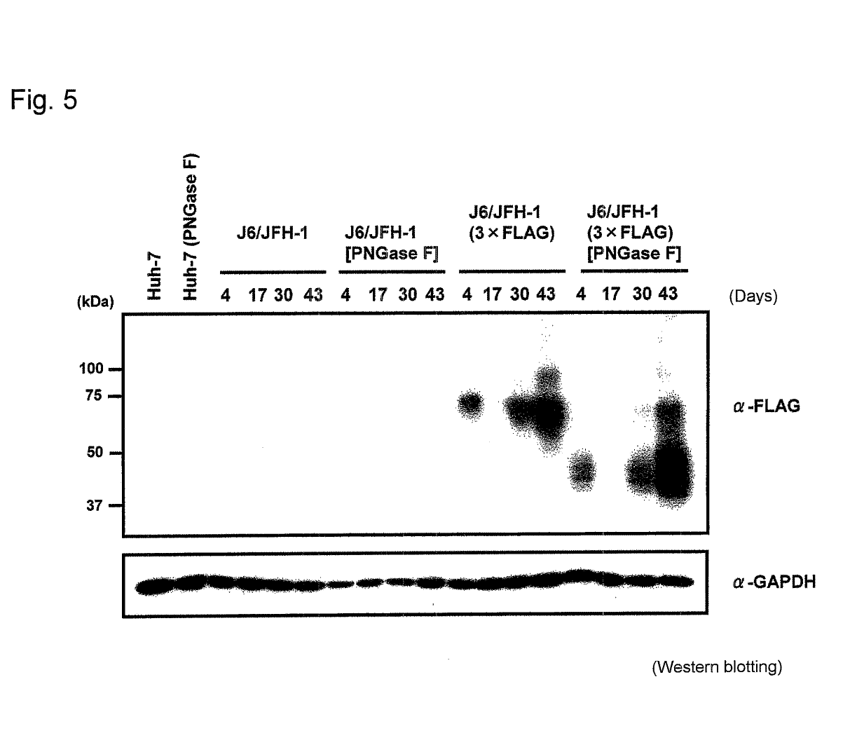

As a result, the molecular mass of the E2 protein 30 and 43 days after the introduction of J6/JFH-1 (3×FLAG) was found to be smaller than that of the E2 protein just when RNA was introduced (FIG. 4). Such change was not observed in the Core proteins. So, these E2 proteins were treated with the deglycosylation enzyme PNGase F, and as a result, such difference in molecular mass was no longer observed (FIG. 5). This suggested that the E2 region of the J6/JFH-1 (3×FLAG) RNA genome might cause a mutation in the genome by repeating subculture of cells and that the site of mutation might be a sugar chain-binding site.

### Example 5

### Analysis of HCV Genome Sequence in Cells Infected with J6/JFH-1 (3×FLAG) Virus Repeatedly Subcultured

To examine an adaptive mutation, which is necessary for the J6/JFH-1 (3×FLAG) virus to have high infectivity, total RNA was extracted from the infected cells 8 days and 39 days after RNA introduction, and the sequence of the HC V genome contained therein was analyzed.

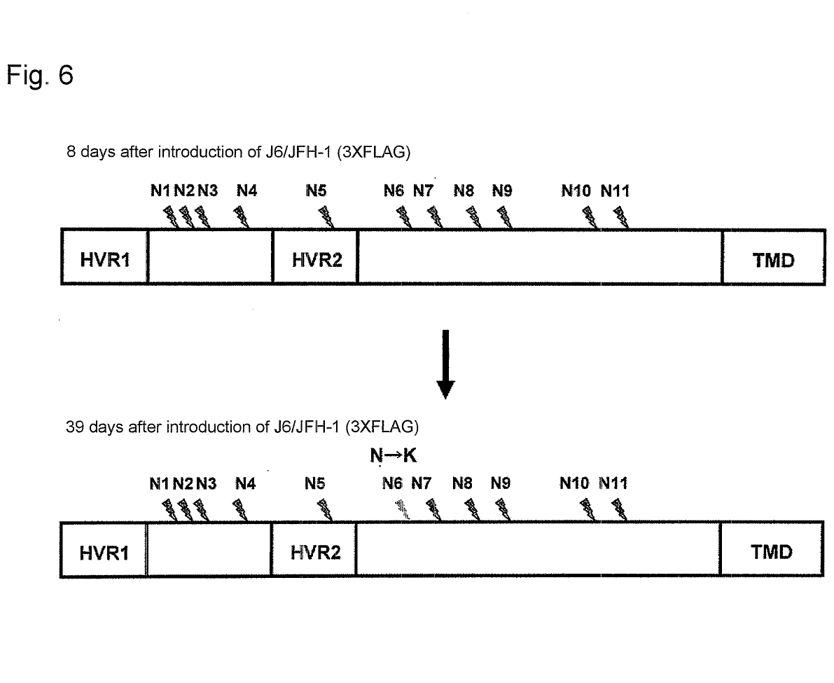

As a result, it was found that asparagine AAU (N), which is a sugar-chain addition site of the E2 region, was substituted with lysine AAA (K), and that, when the sugar chain modification at this site was removed, the molecular mass became smaller than that of the E2 protein at the time of introduction of RNA (FIG. 6). This asparagine corresponds to the 534^{th} amino acid when that the methionine is counted as a first amino acid, the methionine being at the N-terminus in the Core protein of the strain J6CF (GenBank Accession No. AF177036, Yanagi, M. et al., Virology, 262, 1999, pp. 250-263).

### Example 6

### Purification of J6/JFH-1 (3×FLAG) Virus Particles

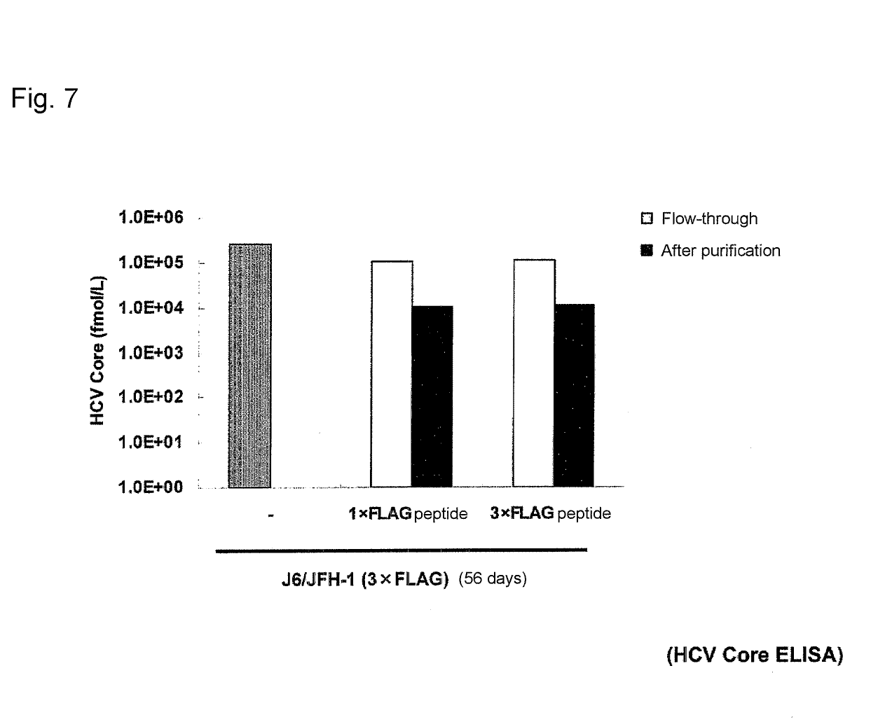

In order to purify J6/JFH-1 (3×FLAG) virus particles, the culture supernatant 56 days after the introduction of J6/JFH-1 (3×FLAG) RNA was used. To 600 μl of this culture supernatant in a container, 500 μl of M2 antibody agarose (Sigma) that had been pretreated with 0.1 M glycine-HCl (pH 3.5) was added, and for mixing, the top and bottom of the container were repeatedly inverted at 4° C. for 2 hours. Flow-through fraction was recovered, while the M2 antibody agarose was washed three times with TBS buffer, and elution was then carried out three times using 200 μl of a 1×FLAG peptide (Sigma) solution or 3×FLAG peptide (Sigma) solution. The amounts of the HCV Core protein in the elution samples were quantified using the HCV antigen ELISA test kit (Ortho). As a result, it was found that elution could be carried out with either of the FLAG peptides, and that purification efficiency was about 5% (FIG. 7).

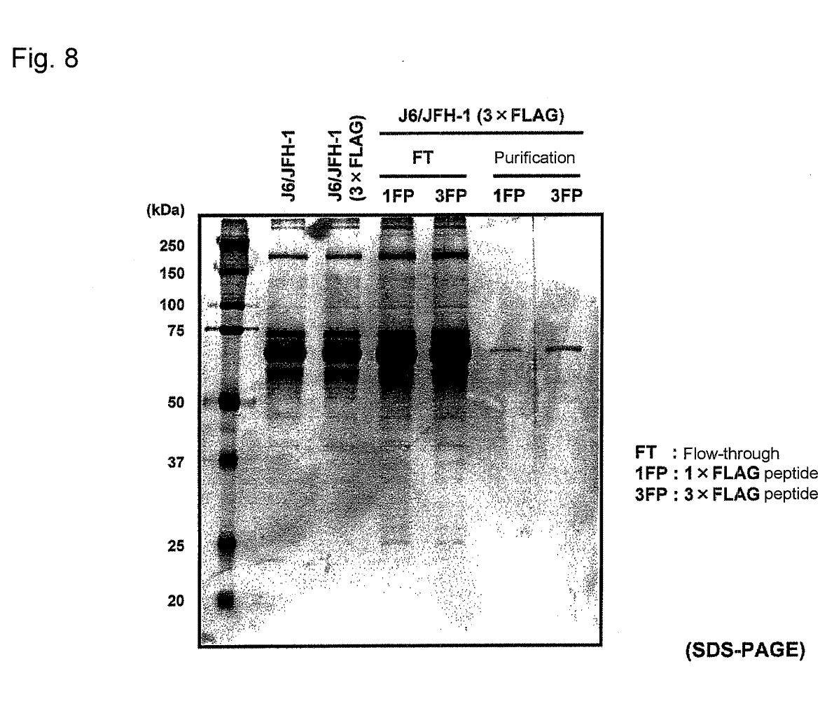

To examine the purity of the elution samples, the elution samples equivalent to 0.1 fmol HCV Core and the unpurified culture supernatant were subjected to SDS-PAGE. Thereafter, silver staining was carried out to confirm the presence of contaminant proteins contained in each solution; however, almost no contaminant proteins were observed in the elution samples (FIG. 8). Thus, it was confirmed that the HCV particles in the elution samples were highly purified.

### Example 7

### Evaluation of Infectivity of Purified J6/JFH-1 (3×FLAG) Virus

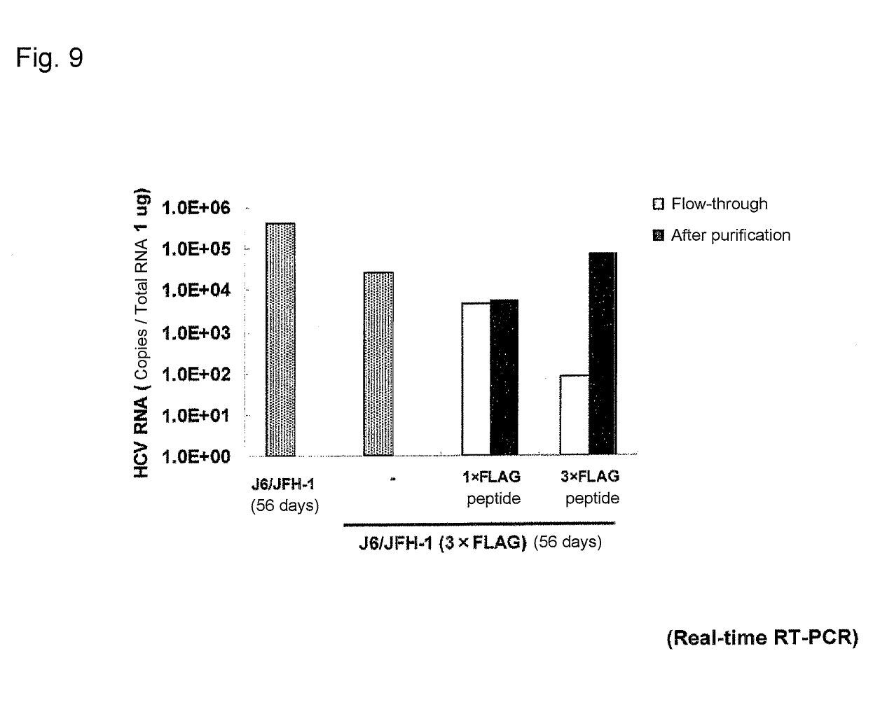

In order to evaluate the infectivity of the J6/JFH-1 (3×FLAG) virus purified in Example 6, infection experiment was carried out using cultured cells. The Huh7 cells were seeded on a 8-well glass chamber at 1×10⁴ cells/well, cultured for 24 hours, and then infected with the virus solution equivalent to 1 fmol HCV Core for 3 hours. After infection, the cells were washed twice with DMEM (Sigma) and cultured for further 4 days. Total RNA was extracted from the cells 4 days later in accordance with the manufacturer's instructions appended to the Trizol Reagent (Invitrogen). The amount of HCV RNA in total RNA was assayed via quantitative RT-PCR. The sense primer S-17 (SEQ ID NO: 13), antisense primer R-19 (SEQ ID NO: 14), and TaqMan probe (SEQ ID NO: 15), which target the 5′-UTR of HCV RNA, were used. The primers, the probe, and extracted total RNA were mixed together to prepare a RT-PCR reaction solution in accordance with the manufacturer's instructions appended to the TaqMan EZ RT-PCR Core Reagents (Applied Biosystems), followed by detection using the 7500 Fast Real-Time PCR System (Applied Biosystems). As a result, the purified J6/JFH-1 (3×FLAG) virus solution was found to have infectivity on the Huh7 cells (FIG. 9). This suggests that the viruses with inserted FLAG-tag sequence in the HVR1 region of HCV E2 can be highly purified and that such purified viruses have infectivity.

### Example 8

### Construction of J6/JFH-1 (1×FLAG) E2 N→K and J6/JFH-1 (3×FLAG) E2 N→K Mutant Plasmids

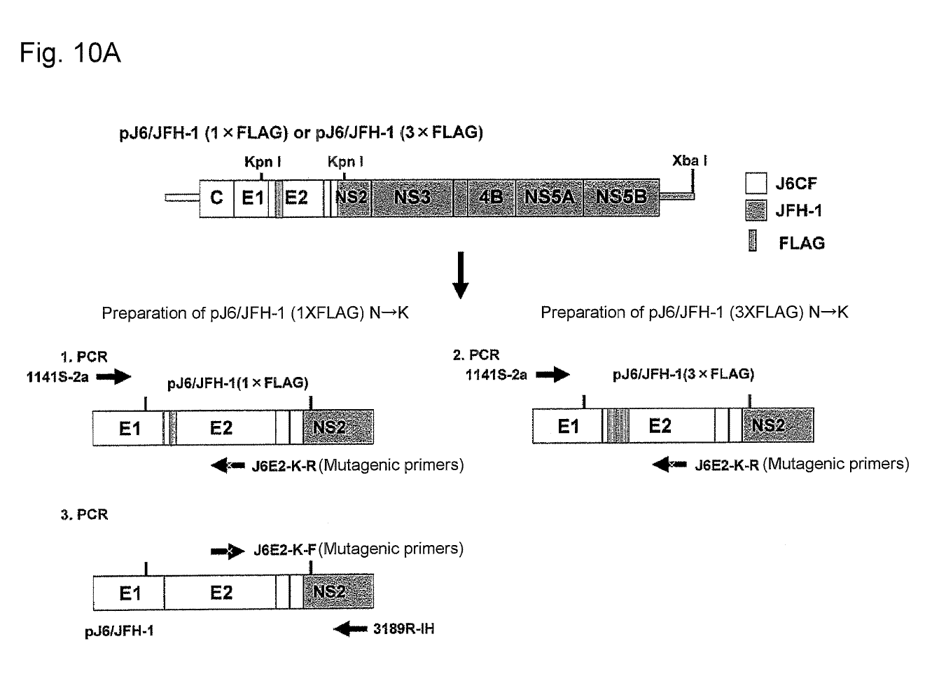

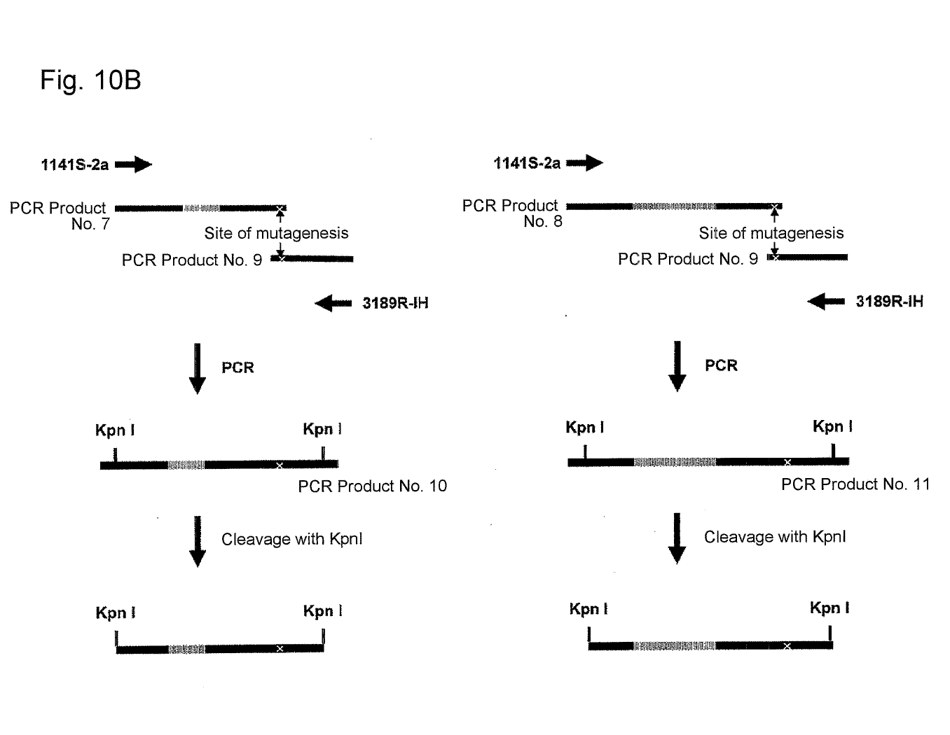

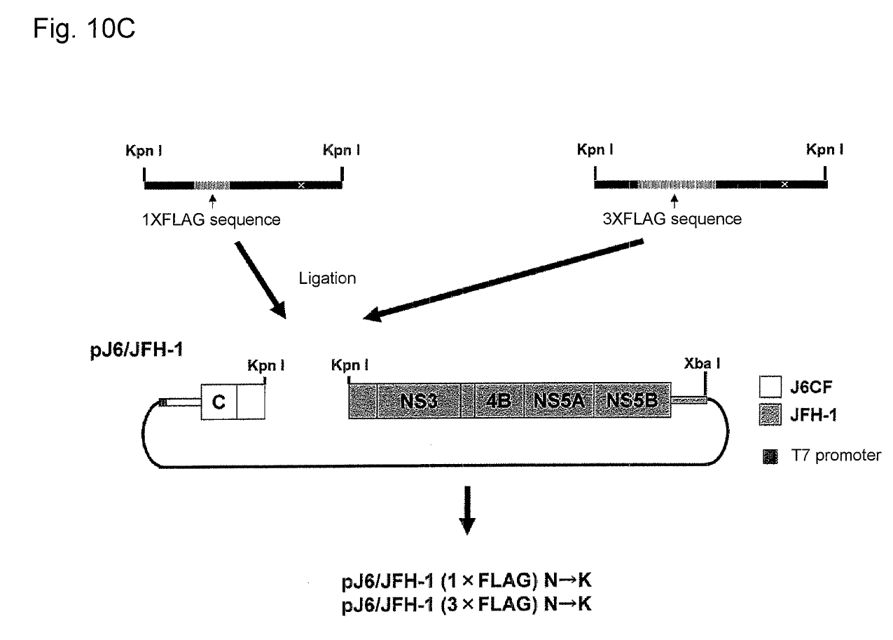

The pJ6/JFH-1 (1×FLAG) N→K and the pJ6/JFH-1 (3×FLAG) N→K plasmids described in Example 5 that have an adaptive mutation necessary for the J6/JFH-1 (3×FLAG) virus to be highly infectious (i.e., a substitution of asparagine AAT (N6) with lysine AAA (K) at the sugar chain addition site of the E2 region) were constructed. The methods for preparing such plasmids are shown in FIG. 10 (A) to (C).

Specifically, to the pJ6/JFH-1 (1×FLAG) as a template were added 10 μl of 10×buffer, 4 μl of 2 mM dNTPs mixture, and 1 μl each of 10 μM primers 1141S-2a (SEQ ID NO: 16) and J6E2-K-R (SEQ ID NO: 17), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles wherein one cycle consists of 98° C. for 10 seconds, 55° C. for 15 seconds, and 72° C. for 1.5 minutes. The resulting PCR product was designated as PCR product No. 7.

Subsequently, to the pJ6/JFH1 (3×FLAG) as a template were added 10 μl of 10×buffer, 4 μl of 2 mM dNTPs mixture, and 1 μl each of 10 μM primers 1141S-2a (SEQ ID NO: 16) and J6E2-K-R (SEQ ID NO: 17), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles wherein one cycle consists of 98° C. for 10 seconds, 55° C. for 15 seconds, and 72° C. for 1.5 minutes. The resulting PCR product was designated as PCR product No. 8.

To the pJ6/JFH-1 as a template were added 10 μl of 10×buffer, 4 μl of 2 mM dNTPs mixture, and 1 μl each of 10 μM primers J6E2-K-F (SEQ ID NO: 18) and 3189R-IH (SEQ ID NO: 19), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles wherein one cycle consists of 98° C. for 10 seconds, 55° C. for 15 seconds, and 72° C. for 1.5 minutes. The resulting PCR product was designated as PCR product No. 9.

Each PCR product was purified and dissolved in 50 μl of H₂O. DNA (1 μl) of the PCR product No. 7 was mixed with DNA (1 μl) of the PCR product No. 9. To the mixture, which was used as a template, were added 10 μl of 10×buffer, 4 μl of the 2 mM dNTPs mixture, and 1 μl each of 10 μM primers 1141S-2a (SEQ ID NO: 16) and 3189R-IH (SEQ ID NO: 19), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles wherein one cycle consists of 98° C. for 10 seconds, 55° C. for 15 seconds, and 72° C. for 2 minutes. The resulting PCR product was designated as PCR product No. 10.

Subsequently, DNA (1 p. 1) of the PCR product No. 8 was mixed with DNA (1 μl) of the PCR product No. 9. To the mixture, which was used as a template, were added 10 μl of 10×buffer, 4 μl of 2 mM dNTPs mixture, and 1 μl each of 10 μM primers 1141S-2a (SEQ ID NO: 16) and 3189R-IH (SEQ ID NO: 19), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles wherein one cycle consists of 98° C. for 10 seconds, 55° C. for 15 seconds, and 72° C. for 2 minutes. The resulting PCR product was designated as PCR product No. 11. Each PCR product was purified and dissolved in 30 μl of H₂O.

The J6/JFH-1 and the purified PCR product No. 10 were separately digested with the KpnI restriction enzyme, and the HCV cDNA fragments were fractionated by agarose gel electrophoresis, followed by purification. The two DNA fragments were mixed with Ligation Mix (TAKARA BIO INC, Japan) to ligate the two DNA fragments. The vector was designated as pJ6/JFH-1 (1×FLAG) N→K. The pJ6/JFH-1 (1×FLAG) N→K comprises a nucleotide sequence that encodes the chimera gene which comprises: in order from 5′-side to 3′-side, the 5′-untranslated region, the Core protein-coding sequence, the E1 protein-coding sequence, the E2 protein-coding sequence, the p7 protein-coding sequence, and a sequence encoding the N-terminal 16 amino acids of the NS2 protein region, of the J6CF strain; and a sequence encoding a region from the N-terminal 17^{th} amino acid to the C-terminus of the NS2 protein region, the NS3 protein-coding sequence, the NS4A protein-coding sequence, the NS4B protein-coding sequence, the NS5A protein-coding sequence, the NS5B protein-coding region, and the 3′-untranslated region, of the JFH-1 strain, wherein the pJ6/JFH-1 (1×FLAG) N→K comprises, in the NS2 HVR1 region, a nucleotide sequence encoding the FLAG peptide sequence and a nucleotide sequence comprising lysine at position 532 when the methionine is counted as a first aminoacid, the methione being at the N-terminus in the Core protein.

The J6/JFH1 and the purified PCR product No. 11 were separately digested with the KpnI restriction enzyme, and each HCV cDNA fragment was fractionated by agarose gel electrophoresis, followed by purification. The two DNA fragments were mixed with Ligation Mix (TAKARA BIO INC, Japan) to ligate the two DNA fragments. The vector was designated as pJ6/JFH-1 (3×FLAG) N→K. The pJ6/JFH-1 (3×FLAG) N→K comprises a nucleotide sequence that encodes the chimera gene which comprises: in order from 5′-side to 3′-side, the 5′-untranslated region, the Core protein-coding sequence, the E1 protein-coding sequence, the E2 protein-coding sequence, the p7 protein-coding sequence, and a sequence encoding the N-terminal 16 amino acids of the NS2 protein region, of the J6CF strain; and a sequence encoding a region from the N-terminal 17^{th} amino acid to the C-terminus of the NS2 protein region, the NS3 protein-coding sequence, the NS4A protein-coding sequence, the NS4B protein-coding sequence, the NS5A protein-coding sequence, the NS5B protein-coding region, and the 3′-untranslated region, of the JFH-1 strain, wherein the pJ6/JFH-1 (3×FLAG) N→K comprises, in the NS2 HVR1 region, a nucleotide sequence encoding 3 repeats of the FLAG peptide sequence and a nucleotide sequence comprising lysine at position 532 when the methionine is counted as a first amino acid, the methionine being at the N-terminus in the Core protein.

The nucleotide sequences of pJ6/JFH-1 (1×FLAG) N→K and pJ6/JFH-1 (3×FLAG) N→K are shown in SEQ ID NO: 20 and SEQ ID NO: 21, respectively, in the Sequence Listing.

### Example 9

### Preparation of J6/JFH-1 (1×FLAG) E2 N→K and J6/JFH-1 (3×FLAG) E2 N→K Viruses

The plasmids prepared in Example 8 were cleaved with XbaI and subjected to phenol/chloroform extraction then to ethanol precipitation. The cleaved plasmids each was used as a template to synthesize HCV RNA using the MEGAscript T7 kit (Ambion).

The Huh7 cells (3×10⁶ cells) and 5 μg of the HCV RNA were suspended in 400 μl of the Cytomix solution (120 mM KCl, 0.15 mM CaCl₂, 10 mM K₂HPO₄/KH₂PO₄, 25 mM Hepes, 2 mM EGTA, 5 mM MgCl₂, 20 mM ATP, and 50 mM glutathione), and transferred into a 4-mm cuvette, and then electroporation was carried out using the Gene Pulser (BioRad) at 260 V and 950 μF. Thereafter, the cells into which the HCV RNA had been introduced were seeded on a 10 cm² dish and then subcultured.

When the cells into which the J6/JFH-1 (1×FLAG) E2 N→K or J6/JFH-1 (3×FLAG) E2 N→K RNA had been introduced were subcultured, each HCV Core protein contained in the culture supernatant was quantified using the HCV antigen ELISA test kit (Ortho), thereby confirming the production of HCV particles. Over the period from the initial stage to the later stage of culture, the amount of HCV Core protein contained in the culture supernatant of cells into which RNA without mutation had been introduced was compared with that contained in the culture supernatant of cells into which RNA with mutation had been introduced. As a result, the latter amount was found to be higher.

### Example 10

### Construction of JFH-1 Plasmid with FLAG-Tag Sequence Inserted into HCV E2 HVR1 Region

In the same manner as in Example 1, an epitope tag sequence was introduced into HVR1 of the genotype 2a strain JFH-1 (GenBank Accession No. AB047639, Kato, T. et al., Gastroenterology, 125, 2003, pp. 1808-1817). The cDNA comprising 1 or 3 repeats of the FLAG sequence was inserted as the epitope. The resulting plasmids are referred to as JFH-1 (1×FLAG) and JFH-1 (3×FLAG), respectively.

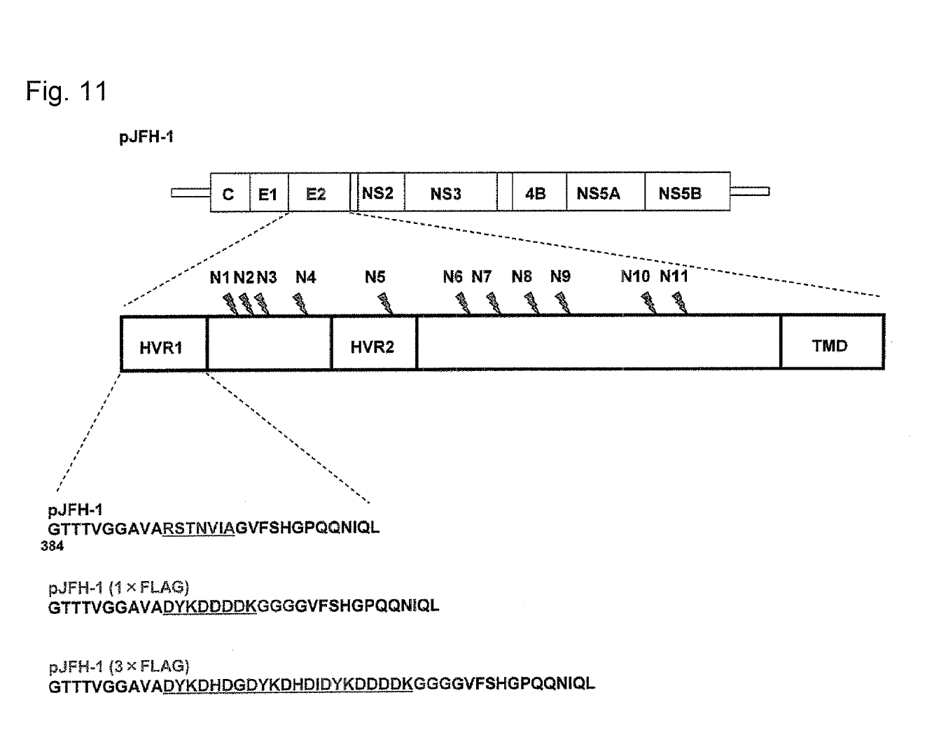

FIG. 11 shows the amino acid sequences, which are in the vicinity of the FLAG-tag insertion sites translated from said genes, starting from the N-terminal amino acid of the E2 protein.

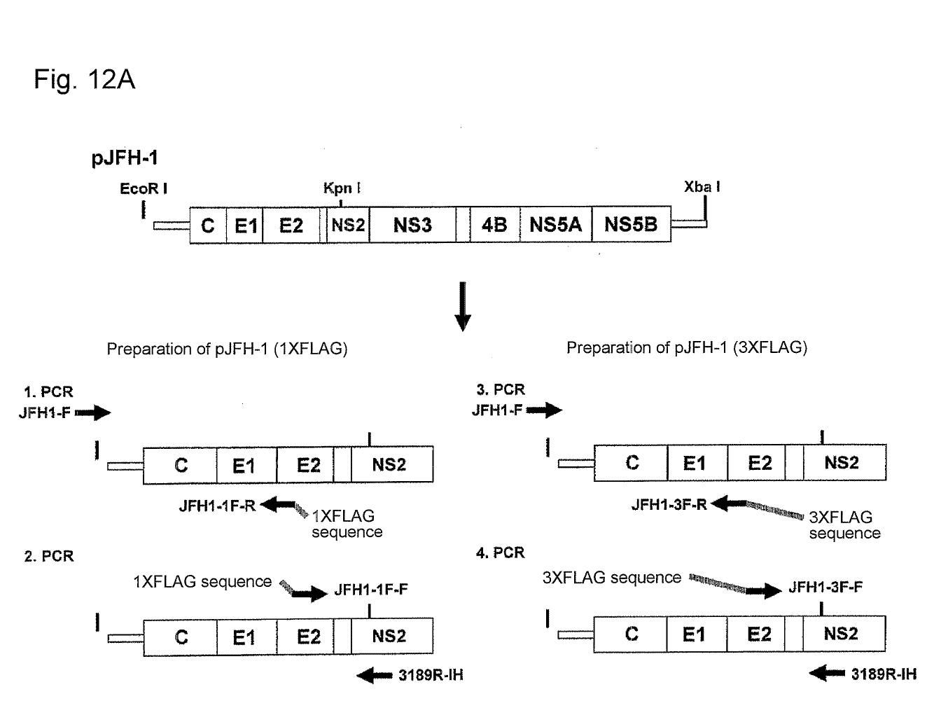

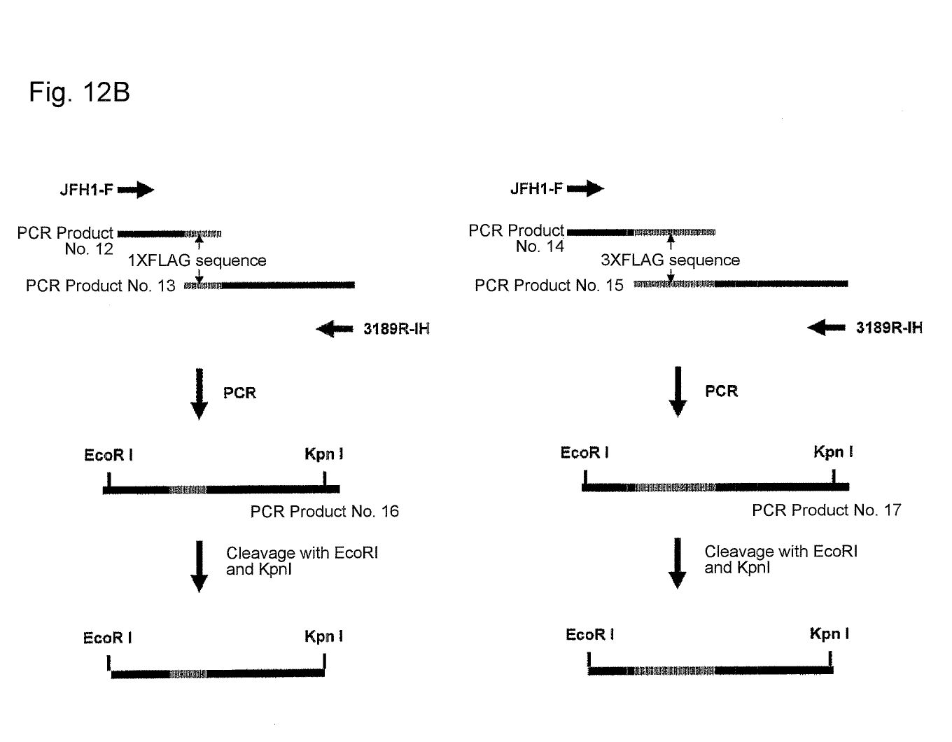

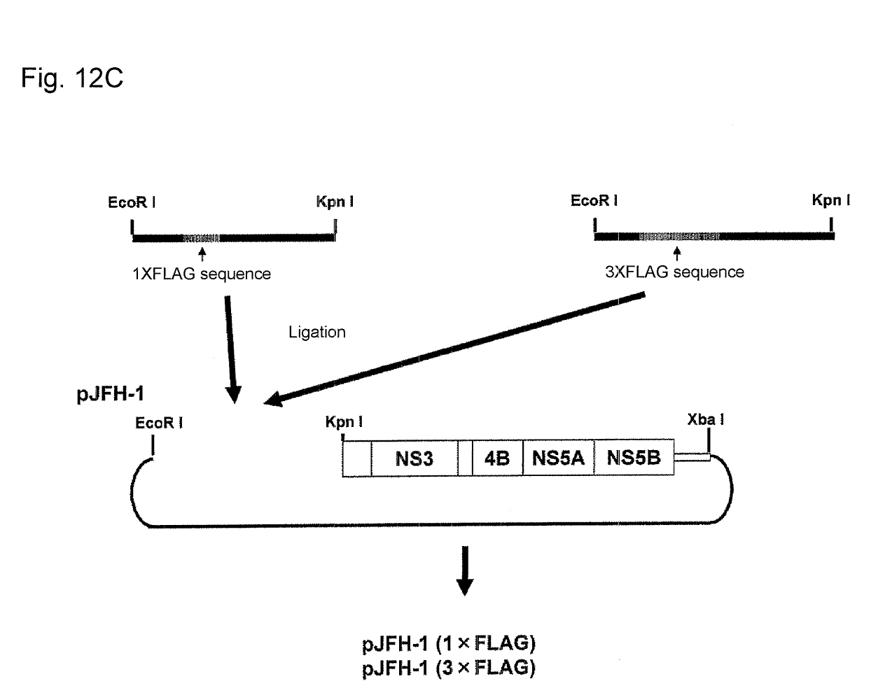

The N-terminal sequence of the E2 protein encoded by pJFH-1 (also referred to as “pJFH1”), the N-terminal sequence of the E2 protein encoded by pJFH-1 (1×FLAG), and the N-terminal sequence of the E2 protein encoded by pJFH-1 (3×FLAG) are shown in SEQ ID NO: 22, SEQ ID NO: 23, and SEQ ID NO: 24, respectively. The methods for preparing these plasmids are shown in FIG. 12 (A) to (C).

Specifically, to the pJFH-1 cDNA as a template were added 10 μl of 10×buffer, 4 μl of 2 mM dNTPs mixture, and 1 μl each of 10 μM primers JFH1-F-R (SEQ ID NO: 25) and JFH1-IF-R (SEQ ID NO: 26), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles, wherein one cycle consists of 98° C. for 10 seconds, 55° C. for 15 seconds, and 72° C. for 30 seconds. The resulting PCR product was designated as PCR product No. 12.

Subsequently, to the pJFH-1 cDNA as a template were added 10 μl of 10×buffer, 4 μl of 2 mM dNTPs mixture, and 1 μl each of 10 μM primers 3189R-IH (SEQ ID NO: 19) and JFH1-1F-F (SEQ ID NO: 27), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles wherein one cycle consists of 98° C. for 10 seconds, 55° C. for 15 seconds, and 72° C. for 1 minute and 30 seconds. The resulting PCR product was designated as PCR product No. 13.

To the JFH-1 cDNA as a template were added 10 μl of 10×buffer, 4 μl of 2 mM dNTPs mixture, and 1 μl each of 10 μM primers JFH1-F (SEQ ID NO: 25) and JFH-1-3F-R (SEQ ID NO: 28), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles wherein one cycle consists of 98° C. for 10 seconds, 55° C. for 15 seconds, and 72° C. for 30 seconds. The resulting PCR product was designated as PCR product No. 14.

To the pJFH-1 cDNA as a template were added 10 μl of 10×buffer, 4 μl of 2 mM dNTPs mixture, and 1 μl each of 10 μM primers 3189R-IH (SEQ ID NO: 19) and JFH1-3F-F (SEQ ID NO: 29), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles wherein one cycle consists of 98° C. for 10 seconds, 55° C. for 15 seconds, and 72° C. for 1.5 minutes. The resulting PCR product was designated as PCR product No. 15.

The PCR products were purified and dissolved in 15 μl of H₂O. DNA (1 μl) of the PCR product No. 12 was mixed with DNA (1 μl) of the PCR product No. 13. To the mixture, which was used as a template, were added 10 μl of 10×buffer, 4 μl of the 2 mM dNTPs mixture, and 1 μl each of 10 μM primers JFH1-F (SEQ ID NO: 25) and 3189R-IH (SEQ ID NO: 19), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles wherein one cycle consists of 98° C. for 10 seconds, 55° C. for 15 seconds, and 72° C. for 2 minutes. The resulting PCR product was designated as PCR product No. 16.

The DNA (1 μl) of the PCR product No. 14 was mixed with DNA (1 μl) of the PCR product No. 15. To the mixture, which was used as a template, were added 10 μl of 10×buffer, 4 μl of 2 mM dNTPs mixture, and 1 μl each of 10 μM primers JFH1-F (SEQ ID NO: 25) and 3189R-IH (SEQ ID NO: 19), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles where one cycle consists of 98° C. for 10 seconds, 55° C. for 15 seconds, and 72° C. for 2 minutes. The resulting PCR product was designated as PCR product No. 17. Each PCR product was purified and dissolved in 30 μl of H₂O.

The pJFH-1 cDNA and the purified PCR product No. 16 were separately digested with the EcoRI and KpnI restriction enzymes, and the HCV cDNA fragments were fractionated by agarose gel electrophoresis, followed by purification. The two DNA fragments were mixed with Ligation Mix (TAKARA BIO INC, Japan) to ligate the two DNA fragments. The vector was designated as pJFH-1 (1×FLAG). The pJFH-1 (1×FLAG) comprises a nucleotide sequence that encodes the gene of the strain JFH1, wherein it comprises, in the E2 HVR1 region, a nucleotide sequence that encodes the FLAG peptide sequence. Subsequently, the pJFH1 cDNA and the purified PCR product No. 17 were separately digested with the EcoRI and KpnI restriction enzymes, and the HCV cDNA fragments were fractionated by agarose gel electrophoresis, followed by purification. The two DNA fragments were mixed with Ligation Mix (TAKARA BIO INC, Japan) to ligate the two DNA fragments. The vector was designated as pJFH-1 (3×FLAG). The pJFH-1 (3×FLAG) comprises a nucleotide sequence that encodes the gene of the strain JFH-1, wherein it comprises, in the E2 HVR1 region, a nucleotide sequence that comprises 3 repeats of the FLAG peptide sequence.

The nucleotide sequences of the pJFH-1 (1×FLAG) and pJFH-1 (3×FLAG) are shown in SEQ ID NO: 30 and SEQ ID NO: 31, respectively, in the Sequence Listing.

### Example 11

### Construction of Plasmid TH/JFH-1 with FLAG-Tag Sequence Inserted in HCV E2 HVR1 Region

Used as a cDNA of HCV genomic RNA was the cDNA of the TH/JFH-1 chimera which comprises a sequence from 5′-UTR to the N-terminal 16^{th} amino acid of NS2 of the genotype 1b strain TH1 (Wakita, T. et al., J. Biol. Chem., 269, 14205-14210, 1994, JP Patent Publication (kokai) No. 2004-179 A) and a sequence from the N-terminal 17^{th} amino acid of NS2 to 3′-UTR of the genotype 2a strain JFH-1 (GenBank Accession No. AB047639, Kato, T. et al., Gastroenterology, 125, 2003, pp. 1808-1817). An epitope tag sequence was introduced into HVR1 of TH/JFH-1. The cDNA comprising 1 or 3 repeats of the FLAG sequence was inserted as the epitope tag. The resulting plasmids are referred to as pTH/JFH-1 (1×FLAG) and pTH/JFH-1 (3×FLAG), respectively.

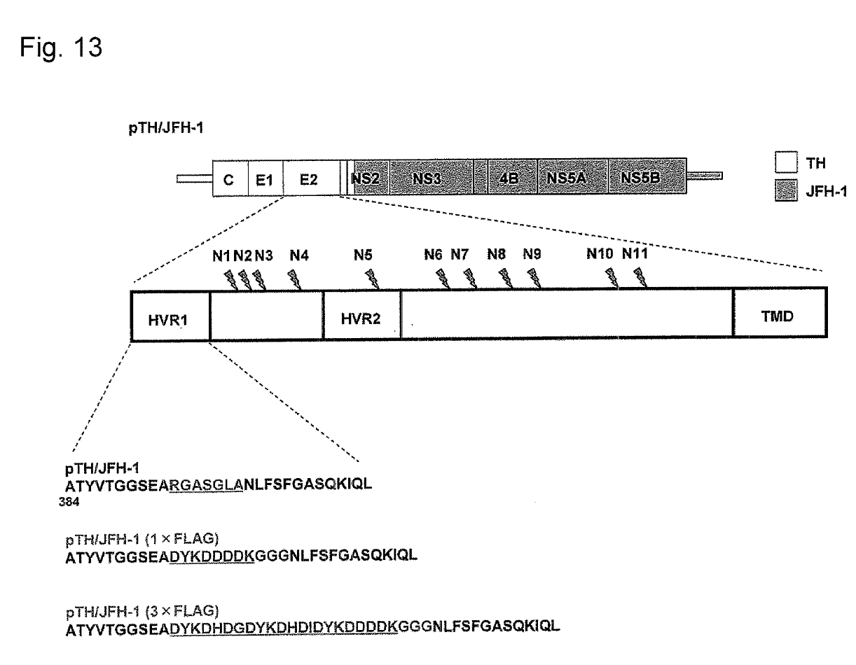

FIG. 13 shows the amino acid sequences which were in the vicinity of the FLAG-tag insertion sites translated from said genes and were from the N-terminal amino acid sequences of the E2 protein.

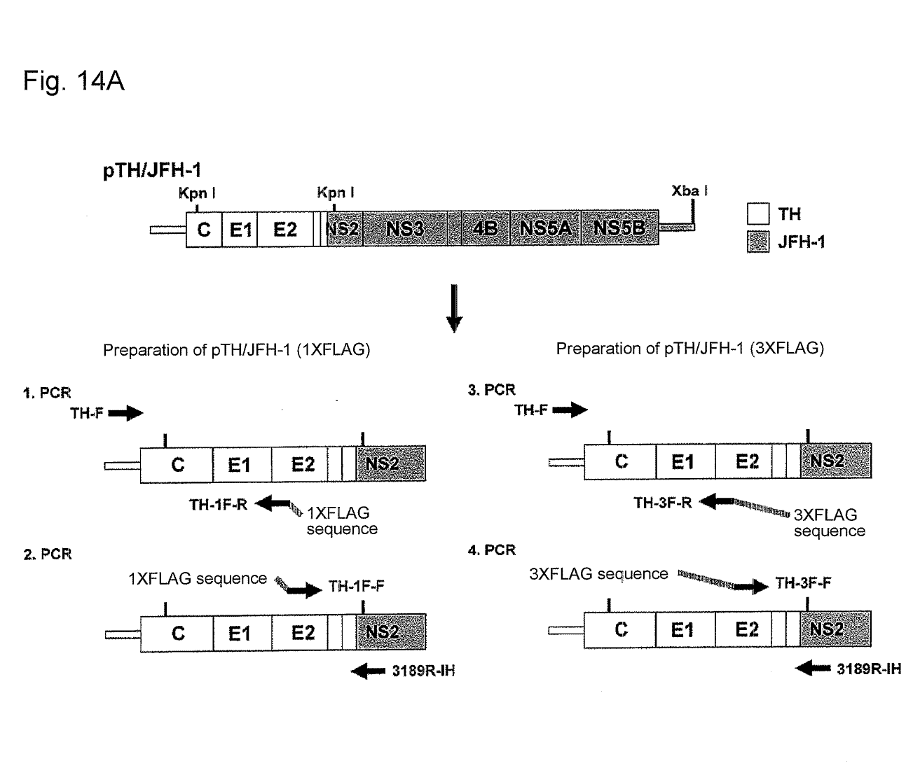

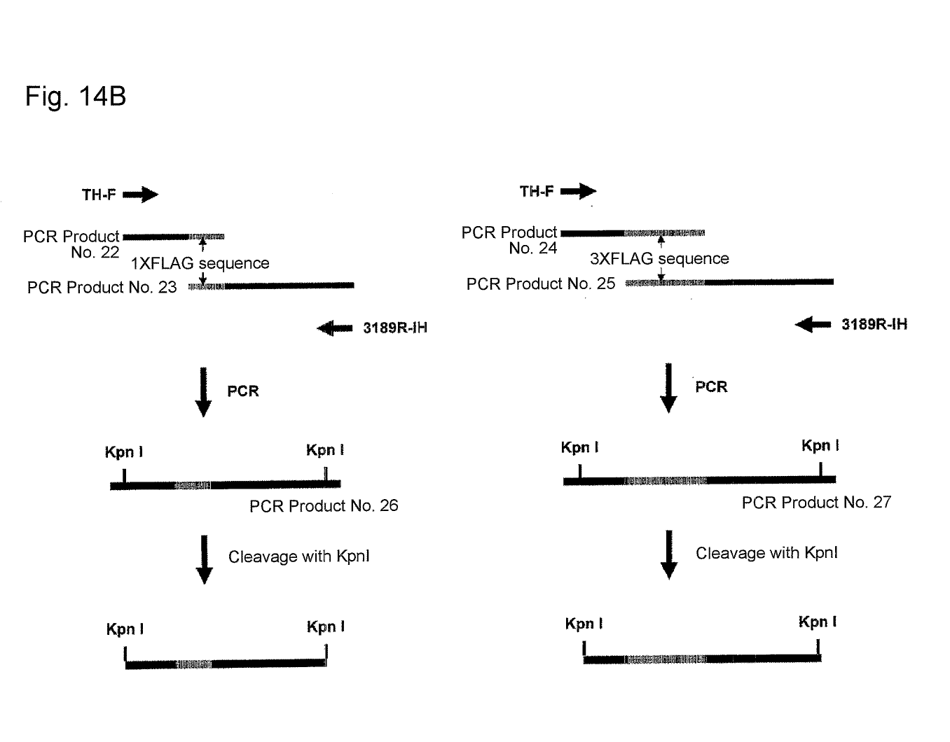

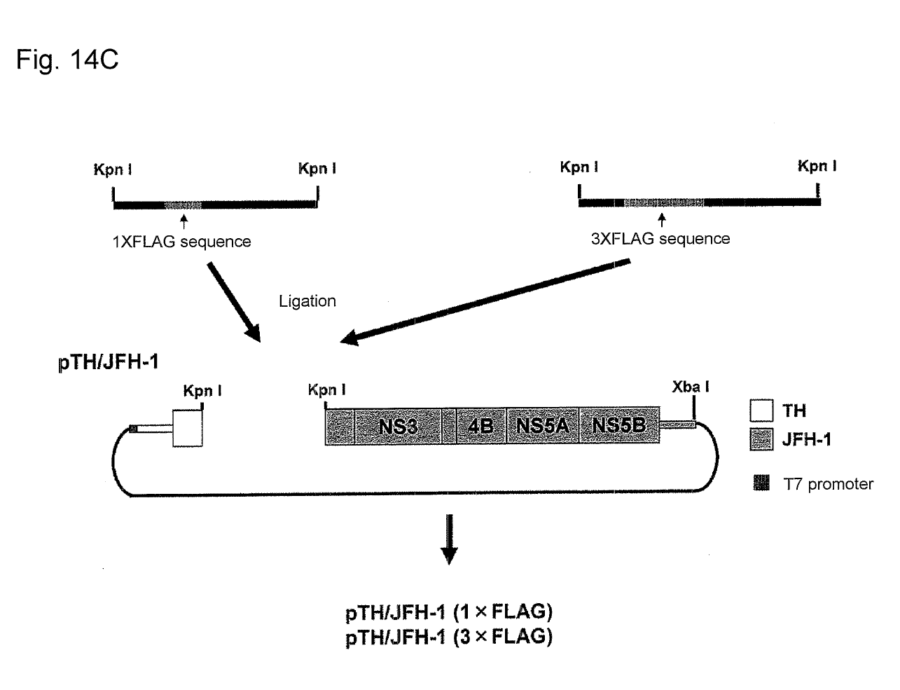

The N-terminal sequence of the E2 protein encoded by pTH/JFH-1, the N-terminal sequence of the E2 protein encoded by pTH/JFH-1 (1×FLAG), and the N-terminal sequence of the E2 protein encoded by pTH/JFH-1 (3×FLAG) are shown in SEQ ID NO: 32, SEQ ID NO: 33, and SEQ ID NO: 34, respectively. The methods for preparing the plasmids are shown in FIG. 14.

The TH/JFH-1 was first prepared. To the pJFH-1 cDNA as a template were added 10 μl of 10×buffer, 4 μl of 2 mM dNTPs mixture, and 1 μl each of 10 μM primers JFH1-A (SEQ ID NO: 43) and JFH1-B (SEQ ID NO: 44), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out to amplify the 5′-untranslated region of JFH-1 and a part of TH Core contained in the primer JFH1-B. PCR was carried out for 30 cycles where one cycle consists of 98° C. for 10 seconds, 55° C. for 15 seconds, and 72° C. for 30 seconds. The resulting PCR product was designated as PCR product No. 18.

Subsequently, to the pTH as a template (Wakita, T. et al., J. Biol. Chem., 269, 14205-14210, 1994 and Moradpour et al., Biochem. Biophys. Res. Commun., 246: 920-924, 1998) were added 10 μl of 10×buffer, 4 μl of 2 mM dNTPs mixture, and 1 μl each of 10 μM primers TH-C (SEQ ID NO: 45) and TH-D (SEQ ID NO: 46), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out to amplify Core to a part of NS2 of TH, a part of the 5′-untranslated region of JFH-1 contained in the primer TH-C, and a part of NS2 of JFH-1 contained in the primer YH-D. PCR was carried out for 30 cycles where one cycle consists of 98° C. for 10 seconds, 55° C. for 15 seconds, and 72° C. for 30 seconds. The resulting PCR product was designated as PCR product No. 19.

To the pJFH-1 as a template were added 10 μl of 10×buffer, 4 μl of t2 mM dNTPs mixture, and 1 μl each of 10 μM primers JFH1-E (SEQ ID NO: 47) and JFH1-F (SEQ ID NO: 48), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out to amplify NS2 and a part of NS3 of JFH-1 and a part of NS2 of TH contained in the primer JFH1-E. PCR was carried out for 30 cycles where one cycle consists of 98° C. for 10 seconds, 55° C. for 15 seconds, and 72° C. for 30 seconds. The resulting PCR product was designated as PCR product No. 20.

PCR was carried out using the PCR product Nos. 18, 19, and 20 and the JFH-A and JFH-E primers to obtain a fragment consisting of the 5′-translated region of the strain JFH-1 to the Core to a part of NS2 of the strain TH, and NS2 to a part of NS3 of JFH-1 (PCR product No. 21).

Subsequently, the pJFH-1 and the PCR product No. 21 were separately treated with EcoRI and SpeI restriction enzymes, and the fragments each was ligated to obtain pTH/JFH-1. The methods for preparing pTH/JFH-1 (1×FLAG) and pTH/JFH-1 (3×FLAG) using this plasmid are shown in FIG. 14 (A) to (C).

Specifically, to the pTH/JFH-1 cDNA as a template were added 10 μl of 10×buffer, 4 μl of 2 mM dNTPs mixture, and 1 μl each of 10 μM primers TH-F (SEQ ID NO: 35) and TH-1F-R (SEQ ID NO: 36), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles where one cycle consists of 98° C. for 10 seconds, 55° C. for 15 seconds, and 72° C. for 30 seconds. The resulting PCR product was designated as PCR product No. 22.

To the pTH/JFH-1 cDNA as a template were added 10 μl of 10×buffer, 4 μl of 2 mM dNTPs mixture, and 1 μl each of 10 μM primers 3189R-IH (SEQ ID NO: 19) and TH-1F-F (SEQ ID NO: 37), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out. PCR was carried out for 30 cycles where one cycle consists of 98° C. for 10 seconds, 55° C. for 15 seconds, and 72° C. for 1.5 minutes. The resulting PCR product was designated as PCR product No. 23.

Subsequently, to the pTH/JFH1 cDNA as a template were added 10 μl of 10×buffer, 4 μl of 2 mM dNTPs mixture, and 1 μl each of 10 μM primers TH-F (SEQ ID NO: 35) and TH-3F-R (SEQ ID NO: 38), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out for 30 cycles where one cycle consists of 98° C. for 10 seconds, 55° C. for 15 seconds, and 72° C. for 30 seconds. The resulting PCR product was designated as PCR product No. 24.

Subsequently, to the pTH/JFH-1 cDNA as a template were added 10 μl of 10×buffer, 4 μl of 2 mM dNTPs mixture, and 1 μl each of 10 μM primers 3189R-IH (SEQ ID NO: 19) and TH-3F-F (SEQ ID NO: 39), which were from the Phusion High-Fidelity DNA Polymerase kit (FINNZYMS), then deionized water was added to bring the total amount to 49.5 μl in the end. Thereafter, 0.5 μl of Phusion DNA Polymerasd (FINNZYMES) was added, and PCR was then carried out for 30 cycles where one cycle consists of 98° C. for 10 seconds, 55° C. for 15 seconds, and 72° C. for 1.5 minutes. The resulting PCR product was designated as PCR product No. 25.

The PCR products were separately purified and dissolved in 15 μl of H₂O. DNA (1 μl) of the PCR product No. 22 was mixed with DNA (1 μl) of the PCR product No. 23. To the mixture, which was used as a template, were added 10 μl of 10×buffer, 4 μl of 2 mM dNTPs mixture, and 1 μl each of 10 μM primers TH-F (SEQ ID NO: 35) and 3189R-IH (SEQ ID NO: 19), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out. PCR was carried out for 30 cycles where one cycle consists of 98° C. for 10 seconds, 55° C. for 15 seconds, and 72° C. for 2 minutes. The resulting PCR product was designated as PCR product No. 26.

Subsequently, DNA (1 μl) of the PCR product No. 24 was mixed with DNA (1 μl) of the PCR product No. 25. To the mixture, which was used as a template, were added 10 μl of 10×buffer, 4 μl of 2 mM dNTPs mixture, and 1 μl each of 10 μM primers TH-F (SEQ ID NO: 35) and 3189R-IH (SEQ ID NO: 19), which were from the Phusion High-Fidelity DNA Polymerase kit (FINNZYMES), then deionized water to bring the total amount to 49.5 μl. Thereafter, 0.5 μl of Phusion DNA Polymerase (FINNZYMES) was added, and PCR was then carried out For 30 cycles where one cycle consists of 98° C. for 10 seconds, 55° C. for 15 seconds, and 72° C. for 2 minutes. The resulting PCR product was designated as PCR product No. 27. The PCR products were purified and dissolved in 30 μl of H₂O.

The pTH/JFH-1 cDNA and the purified PCR product No. 26 were separately digested with the KpnI restriction enzyme, and the HCV cDNA fragments were fractionated by agarose gel electrophoresis, followed by purification. The two DNA fragments were mixed with Ligation Mix (TAKARA BIO INC, Japan) to ligate the two DNA fragments. The vector was designated as pTH/JFH-1 (1×FLAG). The pTH/JFH-1 (1×FLAG) comprises a nucleotide sequence that encodes the gene of the strain TH1 and comprises, in the E2 HVR1 region, a nucleotide sequence that encodes the FLAG peptide sequence.

The pTH/JFH1 cDNA and the purified PCR product No. 27 were separately digested with the KpnI restriction enzyme, and the HCV cDNA fragments were fractionated by agarose gel electrophoresis, followed by purification. The two DNA fragments were mixed with Ligation Mix (TAKARA BIO INC, Japan) to ligate the two DNA fragments. The vector was designated as pTH/JFH-1 (3×FLAG). The pTH/JFH-1 (3×FLAG) comprises a nucleotide sequence that encodes the gene of the strain TH1 and comprises, in the E2 HVR1 region, a nucleotide sequence that comprises 3 repeats of the FLAG peptide sequence.

The nucleotide sequences of pTH/JFH1, pTH/JFH-1 (1×FLAG), and pTH/JFH-1 (3×FLAG) are shown in SEQ ID NO: 40, SEQ ID NO: 41, and SEQ ID NO: 42, respectively, in the Sequence Listing.

All RNAs synthesized from the vectors prepared in the above Examples generated epitope-tagged HCV particles, when introduced into cultured HCV-permissive cells (e.g., Huh7). The culture supernatant containing epitope-tagged HCV particles was allowed to pass through the column filled with a support to which an anti-epitope tagged antibody had been immobilized so as to specifically adsorb the epitope-tagged HCV particles to the supports, and then subjected to elution. Thus, the HCV particles of interest were purified.

### INDUSTRIAL APPLICABILITY

According to the method of producing HCV particles in a cultured cell system and purifying the same of the present invention, HCV particles can be purified more easily at a higher purity than conventional techniques. Since the HCV particles provided by the method of the present invention are of high purity, they are suitable for vaccines for preventing or treating HCV. Further, the epitope-tagged HCV particles of the present invention can be suitably used as tools for inducing antibodies to HCV.

Since the HCV particles provided by the method of the present invention are of high purity, they are suitable for vaccines for preventing or treating HCV. Further, the epitope-tagged HCV particles of the present invention can be suitably used as tools for inducing antibodies to HCV.

All publications, patents, and patent applications cited herein are incorporated herein by reference in their entirety.

Sequence Listing Free Text
SEQ ID NO: 1:
N terminal sequence of E2 protein encoded by
J6/JFH-1
SEQ ID NO: 2:
N terminal sequence of E2 protein encoded by
pJ6/JFH-1 (1XFLAG)
SEQ ID NO: 3:
N terminal sequence of E2 protein encoded by
pJ6/JFH-1 (3XFLAG)
SEQ ID NO: 4:
Primer (E2-F)
SEQ ID NO: 5:
Primer (1F-R)
SEQ ID NO: 6:
Primer (E2-R)
SEQ ID NO: 7:
Primer (1F-F)
SEQ ID NO: 8:
Primer (E2-F)
SEQ ID NO: 9:
Primer (3F-R)
SEQ ID NO: 10:
pJ6/JFH-1
SEQ ID NO: 11:
pJ6/JFH-1 (1XFLAG)
SEQ ID NO: 12:
pJ6/JFH-1 (3XFLAG)
SEQ ID NO: 13:
Primer (S-17)
SEQ ID NO: 14:
Primer (R-19)
SEQ ID NO: 15:
Probe
SEQ ID NO: 16:
Primer (1141S-2a)
SEQ ID NO: 17:
Primer (J6E2-K-R)
SEQ ID NO: 18:
Primer (J6E2-K-F)
SEQ ID NO: 19:
pJ6/JFH-1 (1 × FLAG) N→K
SEQ ID NO: 21:
pJ6/JFH-1 (3 × FLAG) N→K
SEQ ID NO: 22:
N terminal sequence of E2 protein encoded by
pJFH-1
SEQ ID NO: 23:
N terminal sequence of E2 protein encoded by
pJFH-1 (1XFLAG)
SEQ ID NO: 24:
N terminal sequence of E2 protein encoded by
pJFH-1 (3XFLAG)
SEQ ID NO: 25:
Primer (JFH1-F)
SEQ ID NO: 26:
Primer (JFH1-1F-R)
SEQ ID NO: 27:
Primer (JFH1-1F-F)
SEQ ID NO: 28:
Primer (JFH1-3F-R)
SEQ ID NO: 29:
Primer (JFH1-3F-F)
SEQ ID NO: 30:
pJFH-1 (1XFLAG)
SEQ ID NO: 31:
pJFH-1 (3XFLAG)
SEQ ID NO: 32:
N terminal sequence of E2 protein encoded by
pTH/JFH-1
SEQ ID NO: 33:
N terminal sequence of E2 protein encoded by
pTH/JFH-1 (1XFLAG)
SEQ ID NO: 34:
N terminal sequence of E2 protein encoded by
pTH/JFH-1 (3XFLAG)
SEQ ID NO: 35:
Primer (TH-F)
SEQ ID NO: 36:
Primer (TH-1F-R)
SEQ ID NO: 37:
Primer (TH-1F-F)
SEQ ID NO: 38:
Primer (TH-3F-R)
SEQ ID NO: 39:
Primer (TH-3F-F)
SEQ ID NO: 40:
pTH/JFH1
SEQ ID NO: 41:
pTH/JFH1 (1XFLAG)
SEQ ID NO: 42:
pTH/JFH1 (3XFLAG)
SEQ ID NO: 43:
Primer (JFH1-A)
SEQ ID NO: 44:
Primer (JFH1-B)
SEQ ID NO: 45:
Primer (TH-C)
SEQ ID NO: 46:
Primer (TH-D)
SEQ ID NO: 47:
Primer (JFH1-E)
SEQ ID NO: 48:
Primer (JFH1-F)
SEQ ID NO: 49:
Epitope tag peptide (1XFLAG + Linker)
SEQ ID NO: 50:
Epitope tag peptide (3XFLAG + Linker)
SEQ ID NO: 51:
Epitope tag peptide (1XFLAG)
SEQ ID NO: 52:
Epitope tag peptide (3XFLAG)
SEQ ID NO: 53:
Linker
SEQ ID NO: 54:
Linker

1. A nucleic acid comprising (a), (b), or (c) below and comprising a nucleic acid sequence encoding an epitope tag peptide at hypervariable region 1 (HVR1) in an E2 protein coding sequence of the (a), (b), or (c):
(a) the genome of hepatitis C virus (HCV) JFH-1 strain;
(b) a chimeric hepatitis C virus (HCV) genome comprising: 5′-untranslated region, a Core protein coding sequence, an E1 protein coding sequence, an E2 protein coding sequence, and a p7 protein coding sequence of HCV J6CF strain; and a NS2 protein coding sequence, a NS3 protein coding sequence, a NS4A protein coding sequence, a NS4B protein coding sequence, a NS5A protein coding sequence, a NS5B protein coding sequence, and 3′-untranslated region of the HCV JFH-1 strain; or
(c) a chimeric hepatitis C virus (HCV) genome comprising: 5′-untranslated region of the JFH-1 strain; a Core protein coding sequence, an E1 protein coding sequence, an E2 protein coding sequence, and a p7 protein coding sequence of HCV TH1 strain; and a NS2 protein coding sequence, a NS3 protein coding sequence, a NS4A protein coding sequence, a NS4B protein coding sequence, a NS5A protein coding sequence, a NS 5B protein coding sequence, and 3′-untranslated region of the HCV JFH-1 strain.

2. The nucleic acid according to claim 1, wherein the epitope tag peptide comprises the amino acid sequence DYKDDDDK (SEQ ID NO: 51) or DYKDHDGDYKDHDIDYKDDDDK (SEQ ID NO: 52).

3. The nucleic acid according to claim 1 or 2, wherein the epitope tag peptide further comprises a linker sequence.

4. The nucleic acid according to claim 3, wherein the linker sequence comprises GGG sequence (SEQ ID NO: 53), GGGGS sequence (SEQ ID NO: 54), or (GGGGS)₃ (SEQ ID NO: 55) sequence, or a sequence comprising deletion, substitution or addition of 1-10 amino acids in a part of the sequence GGG (SEQ ID NO: 53), GGGGS (SEQ ID NO: 54) or (GGGGS)₃ (SEQ ID NO: 55).

5. The nucleic acid according to claim 1, which is shown in SEQ ID NO: 11, 12, 30, 31, 41, or 42, wherein nucleotide “T” shown in the sequence listing is read as “U” when the nucleic acid is RNA.

6. The nucleic acid according to claim 1, which, when the amino acid subsequent to the end of the sequence of the hypervariable region 1 (HVR1) is designated as an amino acid at position 1, comprises a mutation which is a substitution of asparagine at position 122 or 124 with lysine.

7. The nucleic acid according to claim 6, which is shown in SEQ ID NO: 20 or 21 wherein nucleotide “T” shown in the Sequence Listing is read as “U” when the nucleic acid is RNA.

8. A vector comprising the nucleic acid according to claim 1.

9. An epitope-tagged hepatitis C virus (HCV) particle comprising the nucleic acid according to claim 1 as the genome.

10. A cell comprising the epitope-tagged hepatitis C virus (HCV) particle according to claim 9.

11. The cell according to claim 10, which is Huh7 or a cell line derived therefrom.

12. A method for purifying epitope-tagged hepatitis C virus (HCV) particle comprising:
a step of preparing epitope-tagged HCV particles from the cell according to claim 10;
a step of bringing a solution containing the epitope-tagged HCV particles into contact with an anti-epitope tag antibody-immobilized support and then adsorbing the epitope-tagged HCV particles to the anti-epitope tag antibody-immobilized support; and
a step of releasing the epitope-tagged HCV particles from the anti-epitope tag antibody-immobilized support to obtain the epitope-tagged HCV particles.

13. The method of purification according to claim 12, wherein the releasing step comprises releasing the epitope-tagged HCV particles from the anti-epitope tag antibody-immobilized support using any of a buffer containing epitope tag peptide, a buffer containing no calcium, 0.1 M glycine buffer (pH 2.5-4.0), 1 M arginine HCl buffer (pH 4.0-5.0), or 0.1-1 mM HCl.

14. An hepatitis C virus (HCV) vaccine, which is obtained by inactivating the epitope-tagged HCV particle according to claim 9.

15. An anti-hepatitis C virus (HCV) antibody to the epitope-tagged HCV particle according to claim 9.


SEQUENCE LISTING (55 sequences)

SEQ ID NO: 1 - protein, 51 aa, Artificial Sequence
  Chemically synthesized sequence N terminal sequence of E2 protein encoded in J6/JFH-1
RTHTVGGSAAQTTGRLTSLFDMGPRQKIQLVNTNGSWHINRTANCNDSLHT

SEQ ID NO: 2 - protein, 55 aa, Artificial Sequence
  Chemically synthesized sequence N terminal sequence of E2 protein encoded in pJ6/JFH-1 (1XFLAG)
RTHTVGGSAADYKDDDDKGGGSLFDMGPRQKIQLVNTNGSWHINRTANCNDSLHT

SEQ ID NO: 3 - protein, 69 aa, Artificial Sequence
  Chemically synthesized sequence N terminal sequence of E2 protein encoded in pJ6/JFH-1 (3XFLAG)
RTHTVGGSAADYKDHDGDYKDHDIDYKDDDDKGGGSLFDMGPRQKIQLVNTNGSWHINRTANCNDSLHT

SEQ ID NO: 4 - DNA, 20 bp, Artificial Sequence
  Chemically synthesized sequence primer (E2-F)
cctggtaccatcactggaca

SEQ ID NO: 5 - DNA, 44 bp, Artificial Sequence
  Chemically synthesized sequence primer (1F-R)
cccttgtcatcgtcatccttgtaatccgcggcagaacccccaac

SEQ ID NO: 6 - DNA, 20 bp, Artificial Sequence
  Chemically synthesized sequence primer (E2-R)
tgggtggtacccactcctga

SEQ ID NO: 7 - DNA, 44 bp, Artificial Sequence
  Chemically synthesized sequence primer (1F-F)
aggatgacgatgacaagggaggcggtagcttatttgacatgggc

SEQ ID NO: 8 - DNA, 65 bp, Artificial Sequence
  Chemically synthesized sequence primer (3F-R)
taatcgatgtcatgatctttataatcaccgtcatggtctttgtagtccgcggcagaacccccaac

SEQ ID NO: 9 - DNA, 65 bp, Artificial Sequence
  Chemically synthesized sequence primer (3F-F)
aagatcatgacatcgattacaaggatgacgatgacaagggaggcggtagcttatttgacatgggc

SEQ ID NO: 10 - DNA, 9683 bp, Artificial Sequence
  Chemically synthesized sequence pJ6/JFH-1
acccgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcggagcggtcccagccacgtggaaggcgccagcccatccctaaagatcggcgctccactggcaaatcctggggaaaaccaggatacccctggcccctatacgggaatgagggactcggctgggcaggatggctcctgtccccccgaggttcccgtccctcttggggccccaatgacccccggcataggtcgcgcaacgtgggtaaggtcatcgataccctaacgtgcggctttgccgacctcatggggtacatccctgtcgtgggcgccccgctcggcggcgtcgccagagctctcgcgcatggcgtgagagtcctggaggacggggttaattttgcaacagggaacttacccggttgctccttttctatcttcttgctggccctgctgtcctgcatcaccaccccggtctccgctgccgaagtgaagaacatcagtaccggctacatggtgactaacgactgcaccaatgacagcattacctggcagctccaggctgctgtcctccacgtccccgggtgcgtcccgtgcgagaaagtggggaatgcatctcagtgctggataccggtctcaccgaatgtggccgtgcagcggcccggcgccctcacgcagggcttgcggacgcacatcgacatggttgtgatgtccgccacgctctgctctgccctctacgtgggggacctctgcggtggggtgatgctcgcagcccaaatgttcattgtctcgccgcagcaccactggtttgtccaagactgcaattgctccatctaccctggtaccatcactggacaccgcatggcatgggacatgatgatgaactggtcgcccacggctaccatgatcttggcgtacgcgatgcgtgtccccgaggtcattatagacatcattagcggggctcattggggcgtcatgttcggcttggcctacttctctatgcagggagcgtgggcgaaagtcgttgtcatccttctgttggccgccggggtggacgcgcgcacccatactgttgggggttctgccgcgcagaccaccgggcgcctcaccagcttatttgacatgggccccaggcagaaaatccagctcgttaacaccaatggcagctggcacatcaaccgcaccgccctgaactgcaatgactccttgcacaccggctttatcgcgtctctgttctacacccacagcttcaactcgtcaggatgtcccgaacgcatgtccgcctgccgcagtatcgaggccttccgggtgggatggggcgccttgcaatatgaggataatgtcaccaatccagaggatatgagaccctattgctggcactacccaccaaggcagtgtggcgtggtctccgcgaagactgtgtgtggcccagtgtactgtttcacccccagcccagtggtagtgggcacgaccgacaggcttggagcgcccacttacacgtggggggagaatgagacagatgtcttcctattgaacagcactcgaccaccgctggggtcatggttcggctgcacgtggatgaactcttctggctacaccaagacttgcggcgcaccaccctgccgtactagagctgacttcaacgccagcacggacctgttgtgccccacggactgttttaggaagcatcctgataccacttacctcaaatgcggctctgggccctggctcacgccaaggtgcctgatcgactacccctacaggctctggcattacccctgcacagttaactataccatcttcaaaataaggatgtatgtgggaggggttgagcacaggctcacggctgcatgcaatttcactcgtggggatcgttgcaacttggaggacagagacagaagtcaactgtctcctttgttgcactccaccacggaatgggccattttaccttgctcttactcggacctgcccgccttgtcgactggtcttctccacctccaccaaaacatcgtggacgtacaattcatgtatggcctatcacctgccctcacaaaatacatcgtccgatgggagtgggtaatactcttattcctgctcttagcggacgccagggtttgcgcctgcttatggatgctcatcttgttgggccaggccgaagcagcactagagaagctggtcatcttgcacgctgcgagcgcagctagctgcaatggcttcctatattttgtcatctttttcgtggctgcttggtacatcaagggtcgggtagtccccttagctacctattccctcactggcctgtggtcctttagcctactgctcctagcattgccccaacaggcttatgcttatgacgcatctgtgcatggccagataggagcggctctgctggtaatgatcaccctcttcacactcaccccggggtataagaccctcctcggccagtgtctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgtctaga

SEQ ID NO: 11 - DNA, 9695 bp, Artificial Sequence
  Chemically synthesized sequence pJ6/JFH-1 (1XFLAG)
acccgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcggagcggtcccagccacgtggaaggcgccagcccatccctaaagatcggcgctccactggcaaatcctggggaaaaccaggatacccctggcccctatacgggaatgagggactcggctgggcaggatggctcctgtccccccgaggttcccgtccctcttggggccccaatgacccccggcataggtcgcgcaacgtgggtaaggtcatcgataccctaacgtgcggctttgccgacctcatggggtacatccctgtcgtgggcgccccgctcggcggcgtcgccagagctctcgcgcatggcgtgagagtcctggaggacggggttaattttgcaacagggaacttacccggttgctccttttctatcttcttgctggccctgctgtcctgcatcaccaccccggtctccgctgccgaagtgaagaacatcagtaccggctacatggtgactaacgactgcaccaatgacagcattacctggcagctccaggctgctgtcctccacgtccccgggtgcgtcccgtgcgagaaagtggggaatgcatctcagtgctggataccggtctcaccgaatgtggccgtgcagcggcccggcgccctcacgcagggcttgcggacgcacatcgacatggttgtgatgtccgccacgctctgctctgccctctacgtgggggacctctgcggtggggtgatgctcgcagcccaaatgttcattgtctcgccgcagcaccactggtttgtccaagactgcaattgctccatctaccctggtaccatcactggacaccgcatggcatgggacatgatgatgaactggtcgcccacggctaccatgatcttggcgtacgcgatgcgtgtccccgaggtcattatagacatcattagcggggctcattggggcgtcatgttcggcttggcctacttctctatgcagggagcgtgggcgaaagtcgttgtcatccttctgttggccgccggggtggacgcgcgcacccatactgttgggggttctgccgcggattacaaggatgacgatgacaagggaggcggtagcttatttgacatgggccccaggcagaaaatccagctcgttaacaccaatggcagctggcacatcaaccgcaccgccctgaactgcaatgactccttgcacaccggctttatcgcgtctctgttctacacccacagcttcaactcgtcaggatgtcccgaacgcatgtccgcctgccgcagtatcgaggccttccgggtgggatggggcgccttgcaatatgaggataatgtcaccaatccagaggatatgagaccctattgctggcactacccaccaaggcagtgtggcgtggtctccgcgaagactgtgtgtggcccagtgtactgtttcacccccagcccagtggtagtgggcacgaccgacaggcttggagcgcccacttacacgtggggggagaatgagacagatgtcttcctattgaacagcactcgaccaccgctggggtcatggttcggctgcacgtggatgaactcttctggctacaccaagacttgcggcgcaccaccctgccgtactagagctgacttcaacgccagcacggacctgttgtgccccacggactgttttaggaagcatcctgataccacttacctcaaatgcggctctgggccctggctcacgccaaggtgcctgatcgactacccctacaggctctggcattacccctgcacagttaactataccatcttcaaaataaggatgtatgtgggaggggttgagcacaggctcacggctgcatgcaatttcactcgtggggatcgttgcaacttggaggacagagacagaagtcaactgtctcctttgttgcactccaccacggaatgggccattttaccttgctcttactcggacctgcccgccttgtcgactggtcttctccacctccaccaaaacatcgtggacgtacaattcatgtatggcctatcacctgccctcacaaaatacatcgtccgatgggagtgggtaatactcttattcctgctcttagcggacgccagggtttgcgcctgcttatggatgctcatcttgttgggccaggccgaagcagcactagagaagctggtcatcttgcacgctgcgagcgcagctagctgcaatggcttcctatattttgtcatctttttcgtggctgcttggtacatcaagggtcgggtagtccccttagctacctattccctcactggcctgtggtcctttagcctactgctcctagcattgccccaacaggcttatgcttatgacgcatctgtgcatggccagataggagcggctctgctggtaatgatcaccctcttcacactcaccccggggtataagaccctcctcggccagtgtctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgtctaga

SEQ ID NO: 12 - DNA, 9737 bp, Artificial Sequence
  Chemically synthesized sequence pJ6/JFH-1 (3XFLAG)
acccgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcggagcggtcccagccacgtggaaggcgccagcccatccctaaagatcggcgctccactggcaaatcctggggaaaaccaggatacccctggcccctatacgggaatgagggactcggctgggcaggatggctcctgtccccccgaggttcccgtccctcttggggccccaatgacccccggcataggtcgcgcaacgtgggtaaggtcatcgataccctaacgtgcggctttgccgacctcatggggtacatccctgtcgtgggcgccccgctcggcggcgtcgccagagctctcgcgcatggcgtgagagtcctggaggacggggttaattttgcaacagggaacttacccggttgctccttttctatcttcttgctggccctgctgtcctgcatcaccaccccggtctccgctgccgaagtgaagaacatcagtaccggctacatggtgactaacgactgcaccaatgacagcattacctggcagctccaggctgctgtcctccacgtccccgggtgcgtcccgtgcgagaaagtggggaatgcatctcagtgctggataccggtctcaccgaatgtggccgtgcagcggcccggcgccctcacgcagggcttgcggacgcacatcgacatggttgtgatgtccgccacgctctgctctgccctctacgtgggggacctctgcggtggggtgatgctcgcagcccaaatgttcattgtctcgccgcagcaccactggtttgtccaagactgcaattgctccatctaccctggtaccatcactggacaccgcatggcatgggacatgatgatgaactggtcgcccacggctaccatgatcttggcgtacgcgatgcgtgtccccgaggtcattatagacatcattagcggggctcattggggcgtcatgttcggcttggcctacttctctatgcagggagcgtgggcgaaagtcgttgtcatccttctgttggccgccggggtggacgcgcgcacccatactgttgggggttctgccgcggactacaaagaccatgacggtgattataaagatcatgacatcgattacaaggatgacgatgacaagggaggcggtagcttatttgacatgggccccaggcagaaaatccagctcgttaacaccaatggcagctggcacatcaaccgcaccgccctgaactgcaatgactccttgcacaccggctttatcgcgtctctgttctacacccacagcttcaactcgtcaggatgtcccgaacgcatgtccgcctgccgcagtatcgaggccttccgggtgggatggggcgccttgcaatatgaggataatgtcaccaatccagaggatatgagaccctattgctggcactacccaccaaggcagtgtggcgtggtctccgcgaagactgtgtgtggcccagtgtactgtttcacccccagcccagtggtagtgggcacgaccgacaggcttggagcgcccacttacacgtggggggagaatgagacagatgtcttcctattgaacagcactcgaccaccgctggggtcatggttcggctgcacgtggatgaactcttctggctacaccaagacttgcggcgcaccaccctgccgtactagagctgacttcaacgccagcacggacctgttgtgccccacggactgttttaggaagcatcctgataccacttacctcaaatgcggctctgggccctggctcacgccaaggtgcctgatcgactacccctacaggctctggcattacccctgcacagttaactataccatcttcaaaataaggatgtatgtgggaggggttgagcacaggctcacggctgcatgcaatttcactcgtggggatcgttgcaacttggaggacagagacagaagtcaactgtctcctttgttgcactccaccacggaatgggccattttaccttgctcttactcggacctgcccgccttgtcgactggtcttctccacctccaccaaaacatcgtggacgtacaattcatgtatggcctatcacctgccctcacaaaatacatcgtccgatgggagtgggtaatactcttattcctgctcttagcggacgccagggtttgcgcctgcttatggatgctcatcttgttgggccaggccgaagcagcactagagaagctggtcatcttgcacgctgcgagcgcagctagctgcaatggcttcctatattttgtcatctttttcgtggctgcttggtacatcaagggtcgggtagtccccttagctacctattccctcactggcctgtggtcctttagcctactgctcctagcattgccccaacaggcttatgcttatgacgcatctgtgcatggccagataggagcggctctgctggtaatgatcaccctcttcacactcaccccggggtataagaccctcctcggccagtgtctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgtctaga

SEQ ID NO: 13 - DNA, 17 bp, Artificial Sequence
  Chemically synthesized sequence primer (S-17)
cgggagagccatagtgg

SEQ ID NO: 14 - DNA, 19 bp, Artificial Sequence
  Chemically synthesized sequence primer (R-19)
agtaccacaaggcctttcg

SEQ ID NO: 15 - DNA, 20 bp, Artificial Sequence
  Chemically synthesized sequence probe
ctgcggaaccgtgagtacac

SEQ ID NO: 16 - DNA, 18 bp, Artificial Sequence
  Chemically synthesized sequence primer (1141S-2a)
tgtccgccacgctctgct

SEQ ID NO: 17 - DNA, 21 bp, Artificial Sequence
  Chemically synthesized sequence primer (J6E2-K-R)
atctgtctctttctcccccca

SEQ ID NO: 18 - DNA, 21 bp, Artificial Sequence
  Chemically synthesized sequence primer (J6E2-K-F)
tggggggagaaagagacagat

SEQ ID NO: 19 - DNA, 18 bp, Artificial Sequence
  Chemically synthesized sequence primer (3189R-1H)
ccagtccacctgccaagg

SEQ ID NO: 20 - DNA, 9695 bp, Artificial Sequence
  Chemically synthesized sequence pJ6/JFH-1 (1XFLAG) N->K
acccgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcggagcggtcccagccacgtggaaggcgccagcccatccctaaagatcggcgctccactggcaaatcctggggaaaaccaggatacccctggcccctatacgggaatgagggactcggctgggcaggatggctcctgtccccccgaggttcccgtccctcttggggccccaatgacccccggcataggtcgcgcaacgtgggtaaggtcatcgataccctaacgtgcggctttgccgacctcatggggtacatccctgtcgtgggcgccccgctcggcggcgtcgccagagctctcgcgcatggcgtgagagtcctggaggacggggttaattttgcaacagggaacttacccggttgctccttttctatcttcttgctggccctgctgtcctgcatcaccaccccggtctccgctgccgaagtgaagaacatcagtaccggctacatggtgactaacgactgcaccaatgacagcattacctggcagctccaggctgctgtcctccacgtccccgggtgcgtcccgtgcgagaaagtggggaatgcatctcagtgctggataccggtctcaccgaatgtggccgtgcagcggcccggcgccctcacgcagggcttgcggacgcacatcgacatggttgtgatgtccgccacgctctgctctgccctctacgtgggggacctctgcggtggggtgatgctcgcagcccaaatgttcattgtctcgccgcagcaccactggtttgtccaagactgcaattgctccatctaccctggtaccatcactggacaccgcatggcatgggacatgatgatgaactggtcgcccacggctaccatgatcttggcgtacgcgatgcgtgtccccgaggtcattatagacatcattagcggggctcattggggcgtcatgttcggcttggcctacttctctatgcagggagcgtgggcgaaagtcgttgtcatccttctgttggccgccggggtggacgcgcgcacccatactgttgggggttctgccgcggattacaaggatgacgatgacaagggaggcggtagcttatttgacatgggccccaggcagaaaatccagctcgttaacaccaatggcagctggcacatcaaccgcaccgccctgaactgcaatgactccttgcacaccggctttatcgcgtctctgttctacacccacagcttcaactcgtcaggatgtcccgaacgcatgtccgcctgccgcagtatcgaggccttccgggtgggatggggcgccttgcaatatgaggataatgtcaccaatccagaggatatgagaccctattgctggcactacccaccaaggcagtgtggcgtggtctccgcgaagactgtgtgtggcccagtgtactgtttcacccccagcccagtggtagtgggcacgaccgacaggcttggagcgcccacttacacgtggggggagaaagagacagatgtcttcctattgaacagcactcgaccaccgctggggtcatggttcggctgcacgtggatgaactcttctggctacaccaagacttgcggcgcaccaccctgccgtactagagctgacttcaacgccagcacggacctgttgtgccccacggactgttttaggaagcatcctgataccacttacctcaaatgcggctctgggccctggctcacgccaaggtgcctgatcgactacccctacaggctctggcattacccctgcacagttaactataccatcttcaaaataaggatgtatgtgggaggggttgagcacaggctcacggctgcatgcaatttcactcgtggggatcgttgcaacttggaggacagagacagaagtcaactgtctcctttgttgcactccaccacggaatgggccattttaccttgctcttactcggacctgcccgccttgtcgactggtcttctccacctccaccaaaacatcgtggacgtacaattcatgtatggcctatcacctgccctcacaaaatacatcgtccgatgggagtgggtaatactcttattcctgctcttagcggacgccagggtttgcgcctgcttatggatgctcatcttgttgggccaggccgaagcagcactagagaagctggtcatcttgcacgctgcgagcgcagctagctgcaatggcttcctatattttgtcatctttttcgtggctgcttggtacatcaagggtcgggtagtccccttagctacctattccctcactggcctgtggtcctttagcctactgctcctagcattgccccaacaggcttatgcttatgacgcatctgtgcatggccagataggagcggctctgctggtaatgatcaccctcttcacactcaccccggggtataagaccctcctcggccagtgtctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgtctaga

SEQ ID NO: 21 - DNA, 9737 bp, Artificial Sequence
  Chemically synthesized sequence pJ6/JFH-1 (3XFLAG) N->K
acccgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcggagcggtcccagccacgtggaaggcgccagcccatccctaaagatcggcgctccactggcaaatcctggggaaaaccaggatacccctggcccctatacgggaatgagggactcggctgggcaggatggctcctgtccccccgaggttcccgtccctcttggggccccaatgacccccggcataggtcgcgcaacgtgggtaaggtcatcgataccctaacgtgcggctttgccgacctcatggggtacatccctgtcgtgggcgccccgctcggcggcgtcgccagagctctcgcgcatggcgtgagagtcctggaggacggggttaattttgcaacagggaacttacccggttgctccttttctatcttcttgctggccctgctgtcctgcatcaccaccccggtctccgctgccgaagtgaagaacatcagtaccggctacatggtgactaacgactgcaccaatgacagcattacctggcagctccaggctgctgtcctccacgtccccgggtgcgtcccgtgcgagaaagtggggaatgcatctcagtgctggataccggtctcaccgaatgtggccgtgcagcggcccggcgccctcacgcagggcttgcggacgcacatcgacatggttgtgatgtccgccacgctctgctctgccctctacgtgggggacctctgcggtggggtgatgctcgcagcccaaatgttcattgtctcgccgcagcaccactggtttgtccaagactgcaattgctccatctaccctggtaccatcactggacaccgcatggcatgggacatgatgatgaactggtcgcccacggctaccatgatcttggcgtacgcgatgcgtgtccccgaggtcattatagacatcattagcggggctcattggggcgtcatgttcggcttggcctacttctctatgcagggagcgtgggcgaaagtcgttgtcatccttctgttggccgccggggtggacgcgcgcacccatactgttgggggttctgccgcggactacaaagaccatgacggtgattataaagatcatgacatcgattacaaggatgacgatgacaagggaggcggtagcttatttgacatgggccccaggcagaaaatccagctcgttaacaccaatggcagctggcacatcaaccgcaccgccctgaactgcaatgactccttgcacaccggctttatcgcgtctctgttctacacccacagcttcaactcgtcaggatgtcccgaacgcatgtccgcctgccgcagtatcgaggccttccgggtgggatggggcgccttgcaatatgaggataatgtcaccaatccagaggatatgagaccctattgctggcactacccaccaaggcagtgtggcgtggtctccgcgaagactgtgtgtggcccagtgtactgtttcacccccagcccagtggtagtgggcacgaccgacaggcttggagcgcccacttacacgtggggggagaaagagacagatgtcttcctattgaacagcactcgaccaccgctggggtcatggttcggctgcacgtggatgaactcttctggctacaccaagacttgcggcgcaccaccctgccgtactagagctgacttcaacgccagcacggacctgttgtgccccacggactgttttaggaagcatcctgataccacttacctcaaatgcggctctgggccctggctcacgccaaggtgcctgatcgactacccctacaggctctggcattacccctgcacagttaactataccatcttcaaaataaggatgtatgtgggaggggttgagcacaggctcacggctgcatgcaatttcactcgtggggatcgttgcaacttggaggacagagacagaagtcaactgtctcctttgttgcactccaccacggaatgggccattttaccttgctcttactcggacctgcccgccttgtcgactggtcttctccacctccaccaaaacatcgtggacgtacaattcatgtatggcctatcacctgccctcacaaaatacatcgtccgatgggagtgggtaatactcttattcctgctcttagcggacgccagggtttgcgcctgcttatggatgctcatcttgttgggccaggccgaagcagcactagagaagctggtcatcttgcacgctgcgagcgcagctagctgcaatggcttcctatattttgtcatctttttcgtggctgcttggtacatcaagggtcgggtagtccccttagctacctattccctcactggcctgtggtcctttagcctactgctcctagcattgccccaacaggcttatgcttatgacgcatctgtgcatggccagataggagcggctctgctggtaatgatcaccctcttcacactcaccccggggtataagaccctcctcggccagtgtctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgtctaga

SEQ ID NO: 22 - protein, 30 aa, Artificial Sequence
  Chemically synthesized sequence N terminal sequence of E2 protein encoded in pJFH-1
GTTTVGGAVARSTNVIAGVFSHGPQQNIQL

SEQ ID NO: 23 - protein, 34 aa, Artificial Sequence
  Chemically synthesized sequence N terminal sequence of E2 protein encoded in pJFH-1(1XFLAG)
GTTTVGGAVADYKDDDDKGGGGVFSHGPQQNIQL

SEQ ID NO: 24 - protein, 48 aa, Artificial Sequence
  Chemically synthesized sequence N terminal sequence of E2 protein encoded in pJFH-1 (3˜FLAG)
GTTTVGGAVADYKDHDGDYKDHDIDYKDDDDKGGGGVFSHGPQQNIQL

SEQ ID NO: 25 - DNA, 23 bp, Artificial Sequence
  Chemically synthesized sequence primer(JFH1-F)
ccggaattctaatacgactcact

SEQ ID NO: 26 - DNA, 44 bp, Artificial Sequence
  Chemically synthesized sequence primer(JFH1-1F-R)
cccttgtcatcgtcatccttgtaatctgcaacagcgcctccaac

SEQ ID NO: 27 - DNA, 44 bp, Artificial Sequence
  Chemically synthesized sequence primer(JFH1-1F-F)
aggatgacgatgacaagggaggcggtggcgtgttcagccatggc

SEQ ID NO: 28 - DNA, 65 bp, Artificial Sequence
  Chemically synthesized sequence primer(JFH1-3F-R)
taatcgatgtcatgatctttataatcaccgtcatggtctttgtagtctgcaacagcgcctccaac

SEQ ID NO: 29 - DNA, 65 bp, Artificial Sequence
  Chemically synthesized sequence primer(JFH1-3F-F)
aagatcatgacatcgattacaaggatgacgatgacaagggaggcggtggcgtgttcagccatggc

SEQ ID NO: 30 - DNA, 9690 bp, Artificial Sequence
  Chemically synthesized sequence pJFH-1(1XFLAG)
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacaaatcctaaacctcaaagaaaaaccaaaagaaacaccaaccgtcgcccagaagacgttaagttcccgggcggcggccagatcgttggcggagtatacttgttgccgcgcaggggccccaggttgggtgtgcgcacgacaaggaaaacttcggagcggtcccagccacgtgggagacgccagcccatccccaaagatcggcgctccactggcaaggcctggggaaaaccaggtcgcccctggcccctatatgggaatgagggactcggctgggcaggatggctcctgtccccccgaggctctcgcccctcctggggccccactgacccccggcataggtcgcgcaacgtgggtaaagtcatcgacaccctaacgtgtggctttgccgacctcatggggtacatccccgtcgtaggcgccccgcttagtggcgccgccagagctgtcgcgcacggcgtgagagtcctggaggacggggttaattatgcaacagggaacctacccggtttccccttttctatcttcttgctggccctgttgtcctgcatcaccgttccggtctctgctgcccaggtgaagaataccagtagcagctacatggtgaccaatgactgctccaatgacagcatcacttggcagctcgaggctgcggttctccacgtccccgggtgcgtcccgtgcgagagagtggggaatacgtcacggtgttgggtgccagtctcgccaaacatggctgtgcggcagcccggtgccctcacgcagggtctgcggacgcacatcgatatggttgtgatgtccgccaccttctgctctgctctctacgtgggggacctctgtggcggggtgatgctcgcggcccaggtgttcatcgtctcgccgcagtaccactggtttgtgcaagaatgcaattgctccatctaccctggcaccatcactggacaccgcatggcatgggacatgatgatgaactggtcgcccacggccaccatgatcctggcgtacgtgatgcgcgtccccgaggtcatcatagacatcgttagcggggctcactggggcgtcatgttcggcttggcctacttctctatgcagggagcgtgggcgaaggtcattgtcatccttctgctggccgctggggtggacgcgggcaccaccaccgttggaggcgctgttgcagattacaaggatgacgatgacaagggaggcggtggcgtgttcagccatggccctcagcagaacattcagctcattaacaccaacggcagttggcacatcaaccgtactgccttgaattgcaatgactccttgaacaccggctttctcgcggccttgttctacaccaaccgctttaactcgtcagggtgtccagggcgcctgtccgcctgccgcaacatcgaggctttccggatagggtggggcaccctacagtacgaggataatgtcaccaatccagaggatatgaggccgtactgctggcactaccccccaaagccgtgtggcgtagtccccgcgaggtctgtgtgtggcccagtgtactgtttcacccccagcccggtagtagtgggcacgaccgacagacgtggagtgcccacctacacatggggagagaatgagacagatgtcttcctactgaacagcacccgaccgccgcagggctcatggttcggctgcacgtggatgaactccactggtttcaccaagacttgtggcgcgccaccttgccgcaccagagctgacttcaacgccagcacggacttgttgtgccctacggattgttttaggaagcatcctgatgccacttatattaagtgtggttctgggccctggctcacaccaaagtgcctggtccactacccttacagactctggcattacccctgcacagtcaattttaccatcttcaagataagaatgtatgtagggggggttgagcacaggctcacggccgcatgcaacttcactcgtggggatcgctgcgacttggaggacagggacaggagtcagctgtctcctctgttgcactctaccacggaatgggccatcctgccctgcacctactcagacttacccgctttgtcaactggtcttctccaccttcaccagaacatcgtggacgtacaatacatgtatggcctctcacctgctatcacaaaatacgtcgttcgatgggagtgggtggtactcttattcctgctcttagcggacgccagagtctgcgcctgcttgtggatgctcatcttgttgggccaggccgaagcagcattggagaagttggtcgtcttgcacgctgcgagtgcggctaactgccatggcctcctatattttgccatcttcttcgtggcagcttggcacatcaggggtcgggtggtccccttgaccacctattgcctcactggcctatggcccttctgcctactgctcatggcactgccccggcaggcttatgcctatgacgcacctgtgcacggacagataggcgtgggtttgttgatattgatcaccctcttcacactcaccccggggtataagaccctcctcggccagtgtctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 31 - DNA, 9732 bp, Artificial Sequence
  Chemically synthesized sequence pJFH-1(3XFLAG)
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacaaatcctaaacctcaaagaaaaaccaaaagaaacaccaaccgtcgcccagaagacgttaagttcccgggcggcggccagatcgttggcggagtatacttgttgccgcgcaggggccccaggttgggtgtgcgcacgacaaggaaaacttcggagcggtcccagccacgtgggagacgccagcccatccccaaagatcggcgctccactggcaaggcctggggaaaaccaggtcgcccctggcccctatatgggaatgagggactcggctgggcaggatggctcctgtccccccgaggctctcgcccctcctggggccccactgacccccggcataggtcgcgcaacgtgggtaaagtcatcgacaccctaacgtgtggctttgccgacctcatggggtacatccccgtcgtaggcgccccgcttagtggcgccgccagagctgtcgcgcacggcgtgagagtcctggaggacggggttaattatgcaacagggaacctacccggtttccccttttctatcttcttgctggccctgttgtcctgcatcaccgttccggtctctgctgcccaggtgaagaataccagtagcagctacatggtgaccaatgactgctccaatgacagcatcacttggcagctcgaggctgcggttctccacgtccccgggtgcgtcccgtgcgagagagtggggaatacgtcacggtgttgggtgccagtctcgccaaacatggctgtgcggcagcccggtgccctcacgcagggtctgcggacgcacatcgatatggttgtgatgtccgccaccttctgctctgctctctacgtgggggacctctgtggcggggtgatgctcgcggcccaggtgttcatcgtctcgccgcagtaccactggtttgtgcaagaatgcaattgctccatctaccctggcaccatcactggacaccgcatggcatgggacatgatgatgaactggtcgcccacggccaccatgatcctggcgtacgtgatgcgcgtccccgaggtcatcatagacatcgttagcggggctcactggggcgtcatgttcggcttggcctacttctctatgcagggagcgtgggcgaaggtcattgtcatccttctgctggccgctggggtggacgcgggcaccaccaccgttggaggcgctgttgcagactacaaagaccatgacggtgattataaagatcatgacatcgattacaaggatgacgatgacaagggaggcggtggcgtgttcagccatggccctcagcagaacattcagctcattaacaccaacggcagttggcacatcaaccgtactgccttgaattgcaatgactccttgaacaccggctttctcgcggccttgttctacaccaaccgctttaactcgtcagggtgtccagggcgcctgtccgcctgccgcaacatcgaggctttccggatagggtggggcaccctacagtacgaggataatgtcaccaatccagaggatatgaggccgtactgctggcactaccccccaaagccgtgtggcgtagtccccgcgaggtctgtgtgtggcccagtgtactgtttcacccccagcccggtagtagtgggcacgaccgacagacgtggagtgcccacctacacatggggagagaatgagacagatgtcttcctactgaacagcacccgaccgccgcagggctcatggttcggctgcacgtggatgaactccactggtttcaccaagacttgtggcgcgccaccttgccgcaccagagctgacttcaacgccagcacggacttgttgtgccctacggattgttttaggaagcatcctgatgccacttatattaagtgtggttctgggccctggctcacaccaaagtgcctggtccactacccttacagactctggcattacccctgcacagtcaattttaccatcttcaagataagaatgtatgtagggggggttgagcacaggctcacggccgcatgcaacttcactcgtggggatcgctgcgacttggaggacagggacaggagtcagctgtctcctctgttgcactctaccacggaatgggccatcctgccctgcacctactcagacttacccgctttgtcaactggtcttctccaccttcaccagaacatcgtggacgtacaatacatgtatggcctctcacctgctatcacaaaatacgtcgttcgatgggagtgggtggtactcttattcctgctcttagcggacgccagagtctgcgcctgcttgtggatgctcatcttgttgggccaggccgaagcagcattggagaagttggtcgtcttgcacgctgcgagtgcggctaactgccatggcctcctatattttgccatcttcttcgtggcagcttggcacatcaggggtcgggtggtccccttgaccacctattgcctcactggcctatggcccttctgcctactgctcatggcactgccccggcaggcttatgcctatgacgcacctgtgcacggacagataggcgtgggtttgttgatattgatcaccctcttcacactcaccccggggtataagaccctcctcggccagtgtctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 32 - protein, 30 aa, Artificial Sequence
  Chemically synthesized sequence N terminal sequence of E2 protein encoded in pTH/JFH1
ATYVTGGSEARGASGLANLFSFGASQKIQL

SEQ ID NO: 33 - protein, 34 aa, Artificial Sequence
  Chemically synthesized sequence N terminal sequence of E2 protein encoded in pTH/JFH-1(1XFLAG)
ATYVTGGSEADYKDDDDKGGGNLFSFGASQKIQL

SEQ ID NO: 34 - protein, 48 aa, Artificial Sequence
  Chemically synthesized sequence N terminal sequence of E2 protein encoded in pTH/JFH-1(3XFLAG)
ATYVTGGSEADYKDHDGDYKDHDIDYKDDDDKGGGNLFSFGASQKIQL

SEQ ID NO: 35 - DNA, 20 bp, Artificial Sequence
  Chemically synthesized sequence primer(TH-F)
aggcgacaacctatccccaa

SEQ ID NO: 36 - DNA, 44 bp, Artificial Sequence
  Chemically synthesized sequence primer(TH-1F-R)
cccttgtcatcgtcatccttgtaatcggcttccgacccccccgt

SEQ ID NO: 37 - DNA, 44 bp, Artificial Sequence
  Chemically synthesized sequence primer(TH-1F-F)
aggatgacgatgacaagggaggcggtaacctcttttcatttggg

SEQ ID NO: 38 - DNA, 65 bp, Artificial Sequence
  Chemically synthesized sequence primer(TH-3F-R)
taatcgatgtcatgatctttataatcaccgtcatggtctttgtagtcggcttccgacccccccgt

SEQ ID NO: 39 - DNA, 65 bp, Artificial Sequence
  Chemically synthesized sequence primer(TH-3F-F)
aagatcatgacatcgattacaaggatgacgatgacaagggaggcggtaacctcttttcatttggg

SEQ ID NO: 40 - DNA, 9669 bp, Artificial Sequence
  Chemically synthesized sequence pTH/JFH1
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacgaatcctaaacctcaaagaaaaaccaaacgtaacaccaaccgccgcccacaggacgtcaagttcccgggcggtggccagatcgttggtggagtttacctgttgccgcgcaggggccccaggttgggtgtgcgcgcgactaggaagacttccgagcggtcgcaacctcgtggaaggcgacaacctatccccaaggatcgccgacccgagggcagggcctgggctcagcctgggtacccttggcccctctatggcaacgagggcatggggtgggcaggatggctcctgtcaccccgtggctcccggcctagttggggccccaatgacccccggcgcaggtcgcgtaatttgggtaaagtcatcgatacccttacatgcggcttcgccgacctcatggggtacattccgctcgtcggcgctcccttggggggcgctgccagggccttggcgcatggcgtccgggttctggaggacggcgtgaactatgcaacagggaatctgcccggttgctctttctctatcttcctcttggctctgctgtcctgtctaaccatcccagcttccgcttatgaagtgcgcaacgtgtccggggtgtaccatgtcacgaacgactgctccaactcgagcattgtgtacgagacaggggacatgattatgcacacccctgggtgcgtgccctgtgttcgggagaacaactcctcccgctgctgggcagcgctcactcccacgctcgcggccaggaacgccagcgtccccaccacgacaatacggcgccacgtcgatttgctcgttggggcggctgctttctgctccgctatgtacgtgggggatctctgcggatctgttttcctcgtctcccagttgttcaccttctcgcctcgccggcatgagacagtgcaggactgcaattgttcaatctatcccggccacgtatcaggtcaccgcatggcttgggatatgatgatgaactggtcacctacaacagccctactggtatcgcagttactccggatcccacaagccgtcgtggacatggtggcgggggcccactggggagtcctggcgggccttgcctactattccatggcggggaactgggctaaggttttgattgtgctgctactctttgccggcgttgatggggcgacctacgtgacgggggggtcggaagccagaggggcctctggcttagcaaacctcttttcatttggggcgtctcagaagatccagctcataaataccaacggcagttggcacatcaatagaactgccctgaactgcaatgactccctccacactgggtttcttgccgcgctattctacacacacaaattcaacgcgtccggatgtccagagcgcatggccagctgccgccccattgaagagttcgctcaggggtatggtcccatcacttatgctgagccctccccctcggaccagaggccctattgctggcactacgcgcctcgaccgtgtggtatcatacccgcgtcgcaggtgtgtggtccagtgtactgcttcaccccaagccctgttgtggtggggacgaccgatcgctccggtgcccccacgtataattggggggcgaatgagacggacgtgctgtatctcaacaacacgcggccgccgcaaggcaactggttcggctgcacatggatgaatggcaccgggttcaccaagacgtgcgggggccccccgtgcaacatcggggggggcggcaacaacaacaccttgacctgccccacggactgtttccggaaacaccccgaggccacctacaccaaatgtggttcgggaccttggttgacacctaggtgcatggtcgactacccatacaggctctggcactacccctgcaccgttaactttaccatctttaaggttaggatgtacgtgggaggtgtggagcacaggctcaacgccgcatgcaattggacccgaggagagcgttgtaacttagaggacagggatagatcagagcttagcccgctgctgctgtcaacaacagagtggcaggtgctaccttgttccttcaccaccctaccggctctgtccactggtttgatccatctccaccagaacatcgtggacgtgcaatacctgtacggtatagggtcggcggttgtctcctatgcaatcaaatgggaatatgtcttgttgctcttcctcctcctggcagacgcgcgcgtctgcgcctgcttgtggatgatgctgctgatagctcaagctgaggccgccttagagaacctggtggtcctcaatgcggcgtccctggctggagcgcatggccttctctctttccttgtgttcttctgtgccgcttggtacatcaagggcaggttgatccccggggcggcgtatgctttttacggcgtatggccgctgctcctactcctgctggcgttaccaccacgagcatacgccatggaccgggagatggctgcatcgtgcggaggcgcggtttttgtaggtctggcattcctgaccttgtcaccacactataaggcattcctcgccaagctcctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 41 - DNA, 9681 bp, Artificial Sequence
  Chemically synthesized sequence pTH/JFH1(1XFLAG)
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacgaatcctaaacctcaaagaaaaaccaaacgtaacaccaaccgccgcccacaggacgtcaagttcccgggcggtggccagatcgttggtggagtttacctgttgccgcgcaggggccccaggttgggtgtgcgcgcgactaggaagacttccgagcggtcgcaacctcgtggaaggcgacaacctatccccaaggatcgccgacccgagggcagggcctgggctcagcctgggtacccttggcccctctatggcaacgagggcatggggtgggcaggatggctcctgtcaccccgtggctcccggcctagttggggccccaatgacccccggcgcaggtcgcgtaatttgggtaaagtcatcgatacccttacatgcggcttcgccgacctcatggggtacattccgctcgtcggcgctcccttggggggcgctgccagggccttggcgcatggcgtccgggttctggaggacggcgtgaactatgcaacagggaatctgcccggttgctctttctctatcttcctcttggctctgctgtcctgtctaaccatcccagcttccgcttatgaagtgcgcaacgtgtccggggtgtaccatgtcacgaacgactgctccaactcgagcattgtgtacgagacaggggacatgattatgcacacccctgggtgcgtgccctgtgttcgggagaacaactcctcccgctgctgggcagcgctcactcccacgctcgcggccaggaacgccagcgtccccaccacgacaatacggcgccacgtcgatttgctcgttggggcggctgctttctgctccgctatgtacgtgggggatctctgcggatctgttttcctcgtctcccagttgttcaccttctcgcctcgccggcatgagacagtgcaggactgcaattgttcaatctatcccggccacgtatcaggtcaccgcatggcttgggatatgatgatgaactggtcacctacaacagccctactggtatcgcagttactccggatcccacaagccgtcgtggacatggtggcgggggcccactggggagtcctggcgggccttgcctactattccatggcggggaactgggctaaggttttgattgtgctgctactctttgccggcgttgatggggcgacctacgtgacgggggggtcggaagccgattacaaggatgacgatgacaagggaggcggtaacctcttttcatttggggcgtctcagaagatccagctcataaataccaacggcagttggcacatcaatagaactgccctgaactgcaatgactccctccacactgggtttcttgccgcgctattctacacacacaaattcaacgcgtccggatgtccagagcgcatggccagctgccgccccattgaagagttcgctcaggggtatggtcccatcacttatgctgagccctccccctcggaccagaggccctattgctggcactacgcgcctcgaccgtgtggtatcatacccgcgtcgcaggtgtgtggtccagtgtactgcttcaccccaagccctgttgtggtggggacgaccgatcgctccggtgcccccacgtataattggggggcgaatgagacggacgtgctgtatctcaacaacacgcggccgccgcaaggcaactggttcggctgcacatggatgaatggcaccgggttcaccaagacgtgcgggggccccccgtgcaacatcggggggggcggcaacaacaacaccttgacctgccccacggactgtttccggaaacaccccgaggccacctacaccaaatgtggttcgggaccttggttgacacctaggtgcatggtcgactacccatacaggctctggcactacccctgcaccgttaactttaccatctttaaggttaggatgtacgtgggaggtgtggagcacaggctcaacgccgcatgcaattggacccgaggagagcgttgtaacttagaggacagggatagatcagagcttagcccgctgctgctgtcaacaacagagtggcaggtgctaccttgttccttcaccaccctaccggctctgtccactggtttgatccatctccaccagaacatcgtggacgtgcaatacctgtacggtatagggtcggcggttgtctcctatgcaatcaaatgggaatatgtcttgttgctcttcctcctcctggcagacgcgcgcgtctgcgcctgcttgtggatgatgctgctgatagctcaagctgaggccgccttagagaacctggtggtcctcaatgcggcgtccctggctggagcgcatggccttctctctttccttgtgttcttctgtgccgcttggtacatcaagggcaggttgatccccggggcggcgtatgctttttacggcgtatggccgctgctcctactcctgctggcgttaccaccacgagcatacgccatggaccgggagatggctgcatcgtgcggaggcgcggtttttgtaggtctggcattcctgaccttgtcaccacactataaggcattcctcgccaagctcctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 42 - DNA, 9723 bp, Artificial Sequence
  Chemically synthesized sequence pTH/JFH1(3XFLAG)
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacgaatcctaaacctcaaagaaaaaccaaacgtaacaccaaccgccgcccacaggacgtcaagttcccgggcggtggccagatcgttggtggagtttacctgttgccgcgcaggggccccaggttgggtgtgcgcgcgactaggaagacttccgagcggtcgcaacctcgtggaaggcgacaacctatccccaaggatcgccgacccgagggcagggcctgggctcagcctgggtacccttggcccctctatggcaacgagggcatggggtgggcaggatggctcctgtcaccccgtggctcccggcctagttggggccccaatgacccccggcgcaggtcgcgtaatttgggtaaagtcatcgatacccttacatgcggcttcgccgacctcatggggtacattccgctcgtcggcgctcccttggggggcgctgccagggccttggcgcatggcgtccgggttctggaggacggcgtgaactatgcaacagggaatctgcccggttgctctttctctatcttcctcttggctctgctgtcctgtctaaccatcccagcttccgcttatgaagtgcgcaacgtgtccggggtgtaccatgtcacgaacgactgctccaactcgagcattgtgtacgagacaggggacatgattatgcacacccctgggtgcgtgccctgtgttcgggagaacaactcctcccgctgctgggcagcgctcactcccacgctcgcggccaggaacgccagcgtccccaccacgacaatacggcgccacgtcgatttgctcgttggggcggctgctttctgctccgctatgtacgtgggggatctctgcggatctgttttcctcgtctcccagttgttcaccttctcgcctcgccggcatgagacagtgcaggactgcaattgttcaatctatcccggccacgtatcaggtcaccgcatggcttgggatatgatgatgaactggtcacctacaacagccctactggtatcgcagttactccggatcccacaagccgtcgtggacatggtggcgggggcccactggggagtcctggcgggccttgcctactattccatggcggggaactgggctaaggttttgattgtgctgctactctttgccggcgttgatggggcgacctacgtgacgggggggtcggaagccgactacaaagaccatgacggtgattataaagatcatgacatcgattacaaggatgacgatgacaagggaggcggtaacctcttttcatttggggcgtctcagaagatccagctcataaataccaacggcagttggcacatcaatagaactgccctgaactgcaatgactccctccacactgggtttcttgccgcgctattctacacacacaaattcaacgcgtccggatgtccagagcgcatggccagctgccgccccattgaagagttcgctcaggggtatggtcccatcacttatgctgagccctccccctcggaccagaggccctattgctggcactacgcgcctcgaccgtgtggtatcatacccgcgtcgcaggtgtgtggtccagtgtactgcttcaccccaagccctgttgtggtggggacgaccgatcgctccggtgcccccacgtataattggggggcgaatgagacggacgtgctgtatctcaacaacacgcggccgccgcaaggcaactggttcggctgcacatggatgaatggcaccgggttcaccaagacgtgcgggggccccccgtgcaacatcggggggggcggcaacaacaacaccttgacctgccccacggactgtttccggaaacaccccgaggccacctacaccaaatgtggttcgggaccttggttgacacctaggtgcatggtcgactacccatacaggctctggcactacccctgcaccgttaactttaccatctttaaggttaggatgtacgtgggaggtgtggagcacaggctcaacgccgcatgcaattggacccgaggagagcgttgtaacttagaggacagggatagatcagagcttagcccgctgctgctgtcaacaacagagtggcaggtgctaccttgttccttcaccaccctaccggctctgtccactggtttgatccatctccaccagaacatcgtggacgtgcaatacctgtacggtatagggtcggcggttgtctcctatgcaatcaaatgggaatatgtcttgttgctcttcctcctcctggcagacgcgcgcgtctgcgcctgcttgtggatgatgctgctgatagctcaagctgaggccgccttagagaacctggtggtcctcaatgcggcgtccctggctggagcgcatggccttctctctttccttgtgttcttctgtgccgcttggtacatcaagggcaggttgatccccggggcggcgtatgctttttacggcgtatggccgctgctcctactcctgctggcgttaccaccacgagcatacgccatggaccgggagatggctgcatcgtgcggaggcgcggtttttgtaggtctggcattcctgaccttgtcaccacactataaggcattcctcgccaagctcctgtggtggttgtgctatctcctgaccctgggggaagccatgattcaggagtgggtaccacccatgcaggtgcgcggcggccgcgatggcatcgcgtgggccgtcactatattctgcccgggtgtggtgtttgacattaccaaatggcttttggcgttgcttgggcctgcttacctcttaagggccgctttgacacatgtgccgtacttcgtcagagctcacgctctgataagggtatgcgctttggtgaagcagctcgcggggggtaggtatgttcaggtggcgctattggcccttggcaggtggactggcacctacatctatgaccacctcacacctatgtcggactgggccgctagcggcctgcgcgacttagcggtcgccgtggaacccatcatcttcagtccgatggagaagaaggtcatcgtctggggagcggagacggctgcatgtggggacattctacatggacttcccgtgtccgcccgactcggccaggagatcctcctcggcccagctgatggctacacctccaaggggtggaagctccttgctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgcgcctctagggcggctctcatcgaagaggggcagcggatagccgagatgttgaagtccaagatccaaggcttgctgcagcaggcctctaagcaggcccaggacatacaacccgctatgcaggcttcatggcccaaagtggaacaattttgggccagacacatgtggaacttcattagcggcatccaatacctcgcaggattgtcaacactgccagggaaccccgcggtggcttccatgatggcattcagtgccgccctcaccagtccgttgtcgaccagtaccaccatccttctcaacatcatgggaggctggttagcgtcccagatcgcaccacccgcgggggccaccggctttgtcgtcagtggcctggtgggggctgccgtgggcagcataggcctgggtaaggtgctggtggacatcctggcaggatatggtgcgggcatttcgggggccctcgtcgcattcaagatcatgtctggcgagaagccctctatggaagatgtcatcaatctactgcctgggatcctgtctccgggagccctggtggtgggggtcatctgcgcggccattctgcgccgccacgtgggaccgggggagggcgcggtccaatggatgaacaggcttattgcctttgcttccagaggaaaccacgtcgcccctactcactacgtgacggagtcggatgcgtcgcagcgtgtgacccaactacttggctctcttactataaccagcctactcagaagactccacaattggataactgaggactgccccatcccatgctccggatcctggctccgcgacgtgtgggactgggtttgcaccatcttgacagacttcaaaaattggctgacctctaaattgttccccaagctgcccggcctccccttcatctcttgtcaaaaggggtacaagggtgtgtgggccggcactggcatcatgaccacgcgctgcccttgcggcgccaacatctctggcaatgtccgcctgggctctatgaggatcacagggcctaaaacctgcatgaacacctggcaggggacctttcctatcaattgctacacggagggccagtgcgcgccgaaaccccccacgaactacaagaccgccatctggagggtggcggcctcggagtacgcggaggtgacgcagcatgggtcgtactcctatgtaacaggactgaccactgacaatctgaaaattccttgccaactaccttctccagagtttttctcctgggtggacggtgtgcagatccataggtttgcacccacaccaaagccgtttttccgggatgaggtctcgttctgcgttgggcttaattcctatgctgtcgggtcccagcttccctgtgaacctgagcccgacgcagacgtattgaggtccatgctaacagatccgccccacatcacggcggagactgcggcgcggcgcttggcacggggatcacctccatctgaggcgagctcctcagtgagccagctatcagcaccgtcgctgcgggccacctgcaccacccacagcaacacctatgacgtggacatggtcgatgccaacctgctcatggagggcggtgtggctcagacagagcctgagtccagggtgcccgttctggactttctcgagccaatggccgaggaagagagcgaccttgagccctcaataccatcggagtgcatgctccccaggagcgggtttccacgggccttaccggcttgggcacggcctgactacaacccgccgctcgtggaatcgtggaggaggccagattaccaaccgcccaccgttgctggttgtgctctccccccccccaagaaggccccgacgcctcccccaaggagacgccggacagtgggtctgagcgagagcaccatatcagaagccctccagcaactggccatcaagacctttggccagcccccctcgagcggtgatgcaggctcgtccacgggggcgggcgccgccgaatccggcggtccgacgtcccctggtgagccggccccctcagagacaggttccgcctcctctatgccccccctcgagggggagcctggagatccggacctggagtctgatcaggtagagcttcaacctcccccccagggggggggggtagctcccggttcgggctcggggtcttggtctacttgctccgaggaggacgataccaccgtgtgctgctccatgtcatactcctggaccggggctctaataactccctgtagccccgaagaggaaaagttgccaatcaaccctttgagtaactcgctgttgcgataccataacaaggtgtactgtacaacatcaaagagcgcctcacagagggctaaaaaggtaacttttgacaggacgcaagtgctcgacgcccattatgactcagtcttaaaggacatcaagctagcggcttccaaggtcagcgcaaggctcctcaccttggaggaggcgtgccagttgactccaccccattctgcaagatccaagtatggattcggggccaaggaggtccgcagcttgtccgggagggccgttaaccacatcaagtccgtgtggaaggacctcctggaagacccacaaacaccaattcccacaaccatcatggccaaaaatgaggtgttctgcgtggaccccgccaaggggggtaagaaaccagctcgcctcatcgtttaccctgacctcggcgtccgggtctgcgagaaaatggccctctatgacattacacaaaagcttcctcaggcggtaatgggagcttcctatggcttccagtactcccctgcccaacgggtggagtatctcttgaaagcatgggcggaaaagaaggaccccatgggtttttcgtatgatacccgatgcttcgactcaaccgtcactgagagagacatcaggaccgaggagtccatataccaggcctgctccctgcccgaggaggcccgcactgccatacactcgctgactgagagactttacgtaggagggcccatgttcaacagcaagggtcaaacctgcggttacagacgttgccgcgccagcggggtgctaaccactagcatgggtaacaccatcacatgctatgtgaaagccctagcggcctgcaaggctgcggggatagttgcgcccacaatgctggtatgcggcgatgacctagtagtcatctcagaaagccaggggactgaggaggacgagcggaacctgagagccttcacggaggccatgaccaggtactctgcccctcctggtgatccccccagaccggaatatgacctggagctaataacatcctgttcctcaaatgtgtctgtggcgttgggcccgcggggccgccgcagatactacctgaccagagacccaaccactccactcgcccgggctgcctgggaaacagttagacactcccctatcaattcatggctgggaaacatcatccagtatgctccaaccatatgggttcgcatggtcctaatgacacacttcttctccattctcatggtccaagacaccctggaccagaacctcaactttgagatgtatggatcagtatactccgtgaatcctttggaccttccagccataattgagaggttacacgggcttgacgccttttctatgcacacatactctcaccacgaactgacgcgggtggcttcagccctcagaaaacttggggcgccacccctcagggtgtggaagagtcgggctcgcgcagtcagggcgtccctcatctcccgtggagggaaagcggccgtttgcggccgatatctcttcaattgggcggtgaagaccaagctcaaactcactccattgccggaggcgcgcctactggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 43 - DNA, 18 bp, Artificial Sequence
  Chemically synthesized sequence primer (JFH1-A)
tgtaaaacgacggccagt

SEQ ID NO: 44 - DNA, 40 bp, Artificial Sequence
  Chemically synthesized sequence primer (JFH1-B)
ggtttaggattcgtgctcatggtgcacggtctacgagacc

SEQ ID NO: 45 - DNA, 40 bp, Artificial Sequence
  Chemically synthesized sequence primer (TH-C)
ggtctcgtagaccgtgcaccatgagcacgaatcctaaacc

SEQ ID NO: 46 - DNA, 40 bp, Artificial Sequence
  Chemically synthesized sequence primer (TH-D)
agatagcacaaccaccacaggagcttggcgaggaatgcct

SEQ ID NO: 47 - DNA, 40 bp, Artificial Sequence
  Chemically synthesized sequence primer (JFH1-E)
aggcattcctcgccaagctcctgtggtggttgtgctatct

SEQ ID NO: 48 - DNA, 20 bp, Artificial Sequence
  Chemically synthesized sequence primer (JFH1-F)
cagctaccgaggggttaagc

SEQ ID NO: 49 - protein, 11 aa, Artificial Sequence
  Chemically synthesized sequence Epitopetag peptide (1XFLAG+Linker)
DYKDDDDKGGG

SEQ ID NO: 50 - protein, 25 aa, Artificial Sequence
  Chemically synthesized sequence Epitopetag peptide (3XFLAG+Linker)
DYKDHDGDYKDHDIDYKDDDDKGGG

SEQ ID NO: 51 - protein, 8 aa, Artificial Sequence
  Chemically synthesized sequence Epitopetag peptide (1XFLAG)
DYKDDDDK

SEQ ID NO: 52 - protein, 22 aa, Artificial Sequence
  Chemically synthesized sequence Epitopetag peptide (3XFLAG)
DYKDHDGDYKDHDIDYKDDDDK

SEQ ID NO: 53 - protein, 3 aa, Artificial Sequence
  Chemically synthesized sequence Linker
GGG

SEQ ID NO: 54 - protein, 5 aa, Artificial Sequence
  Chemically synthesized sequence Linker
GGGGS

SEQ ID NO: 55 - protein, 15 aa, Artificial Sequence
  Chemically synthesized sequence Linker
GGGGSGGGGSGGGGS